(19) United States
(12) Patent Application Publication
(10) Pub. No.: US 2025/0277176 A1
(43) Pub. Date: Sep. 4, 2025

(54) MICROPHYSIOLOGICAL SYSTEM AND METHOD FOR CONTINUOUS METABOLIC MEASUREMENT OF EPITHELIAL TISSUE

(71) Applicant: Colorado State University Research Foundation, Fort Collins, CO (US)

(72) Inventors: Thomas Wei Chen, Fort Collins, CO (US); Ryan Way, Fort Collins, CO (US)

(21) Appl. No.: 19/066,418

(22) Filed: Feb. 28, 2025

Related U.S. Application Data
(60) Provisional application No. 63/559,592, filed on Feb. 29, 2024.

(51) Int. Cl. C12M 3/00; C12M 1/00; C12M 1/34; C12M 1/36

(52) U.S. Cl. CPC C12M 21/08; C12M 23/34; C12M 23/46; C12M 29/00; C12M 41/26; C12M 41/34; C12M 41/38; C12M 41/48

(57) ABSTRACT

An exemplary microphysiological system and method are disclosed comprising a microfluidic chamber designed to hold live tissue explants and provide a desired microphysiological environment for the tissue to keep it alive and thriving for an extended period of time to which observations and stimuli can be applied to the tissue. The exemplary microphysiological system and method are self-contained and scalable to allow multiple chambers to operate in parallel in high throughput capacity.

### CROSS-REFERENCE TO RELATED APPLICATIONS

This application claims priority to and the benefit of U.S. Provisional Application No. 63/559,592, titled “MICROPHYSIOLOGICAL SYSTEM AND METHOD FOR CONTINUOUS METABOLIC MEASUREMENT OF EPITHELIAL TISSUE,” filed on Feb. 29, 2024, the content of which is hereby incorporated by reference herein in its entirety.

### STATEMENT REGARDING FEDERALLY SPONSORED RESEARCH OR DEVELOPMENT

This invention was made with government support under grant no. 0841259 and grant no. 1450032 awarded by the National Science Foundation. The government has certain rights in the invention.

### BACKGROUND

Tissue barriers are made up of one or more cell types that collectively form a distinct boundary between one physiological environment and another. Typically, these barriers separate the body or sensitive organs or tissues from exposure to substrates of the external environment, including pathogens or drugs.

Epithelial cells create barriers that protect different components in the body from their external environment. The gut, for example, carries bacteria and other infectious agents. A healthy gut epithelial barrier can prevent certain substances from accessing the underlying lamina propria while maintaining the ability to digest and absorb nutrients. Increased gut barrier permeability, better known as leaky gut, has been linked to several chronic inflammatory diseases. Yet understanding the cause of leaky gut and developing effective interventions are still elusive due to the challenges associated with maintaining the tissue's physiological environment while elucidating cellular functions under various stimuli ex vivo.

Drug testing or discovery relies on animal models prior to human testing to evaluate the effects of a therapeutic compound on a global scale of the animal. There is a lack or incomplete understanding of the long-term effects or exposure of drugs on an animal at the cellular or tissue level, particularly for epithelial and various barrier cells.

There is a benefit to providing viability and function of epithelial tissue and other barrier cell tissue over an extended period of time.

### SUMMARY

An exemplary microphysiological system and method are disclosed comprising a microfluidic chamber designed to hold live tissue explants and provide a desired microphysiological environment for the tissue to keep it alive and thriving for an extended period of time to which observations and stimuli can be applied to the tissue. The exemplary microphysiological system and method are self-contained and scalable to allow multiple chambers to operate in parallel in high throughput capacity.

A desired microphysiological environment refers to an artificially created incubation environment that is physiologically self-contained and that either provides a realistic microphysiological environment for barrier cell tissue (e.g., epithelial tissue, among others described herein) to live and grow or provides an artificial one that nevertheless promotes tissue growth. To generate the desired microphysiological environment, the microfluidic chamber is configured to provide nutrient flow across the tissue and chamber observed to create necessary oxygen gradients across the tissue barrier to maintain and promote tissue viability while readily removing artifacts such as air bubbles and cellular waste that are byproducts of the tissue's metabolic activities or byproducts from the measurement instrumentation. The microfluidic chamber further includes anchoring structures for tissue fixation in the microphysiological environment.

The microfluidic chamber includes integrated electrodes that couple to on-board supporting electronics of the microphysiological system to be instrumentally self-contained, in addition to being physiologically self-contained, enabling scalability and parallel operation of multiple systems for high throughput measurements. The exemplary microphysiological system and method can be employed, in a self-contained manner, for continuous trans-barrier-cell electrical resistance measurement (e.g., transepithelial electrical resistance (TEER) measurement), continuous real-time barrier permeability recording or assessment of the tissue, or continuous metabolic measurements of the tissue over an extended duration that requires minimal or no supervision or intervention.

As used herein, the term “tissue” refers to animal or human subject tissue, such as, and not limited to, muscle tissue (cardiac muscle tissue, skeletal muscle tissue, smooth muscle tissue, epithelial tissue (skin tissue, organ tissue, reproductive tissue), connective tissue (tendon, bone, fat), endothelial tissue, brain tissue, and/or the like. Barrier cell tissue refers to one or more cell types that collectively form a distinct boundary between one physiological environment and another, e.g., to separate or protect the body or sensitive organs or tissues from exposure to substrates of the external environment.

In another implementation, the device is further configured with pairs of pH, O2, glucose, lactate sensors, and/or other sensors described, herein at each respective side of the tissue sample, including at least a first side and a second side of the tissue sample, that are configured to measure metabolites in the media in order to determine a difference in metabolic activity.

A study was conducted to develop and evaluate embodiments of the exemplary microphysiological system and method. Experimental results demonstrated that the system of the study can maintain tissue viability for up to at least 72 hours. The results also show that the custom-built and integrated Trans-Epithelial Electrical Resistance (TEER) sensors, as an example of trans-barrier cell electrical resistance sensors, were sufficiently sensitive to distinguish differing levels of barrier permeability when treated with collagenase and low pH media compared to control. Permeability variations in tissue explants from different positions in the intestinal tract were also investigated using TEER, revealing their disparities in permeability. Finally, the results also quantitatively determine the effect of the muscle layer on total epithelial resistance.

The exemplary system and method can be used for general barrier-cell (e.g., epithelial cell) research, drug discovery and testing on barrier-cell tissue (e.g., epithelial tissue, among others described herein), toxin evaluations on barrier-cell tissue (e.g., epithelial tissue, among others described herein), and barrier-cell tissue barrier evaluation, among other applications described or referenced herein.

In an aspect, a device is disclosed (e.g., for assessing a tissue sample and for receiving a media during such assessment) comprising a first chamber body; a second chamber body having a correspondence to the first chamber body and is configured to fixably couple to the first chamber body to form an assembled chamber, wherein the first chamber body defines a first elongated-curved internal structure, and wherein the second chamber body defines a second elongated-curved internal structure that, together with the first internal structure, defines an elongated-curved chamber volume (e.g., oval or teardrop shape) to hold a tissue sample and to promote laminar flow when media is flowing therethrough, wherein the first chamber body and the second chamber body each define (i) a first fluidic channel having smooth flow paths from a first inlet to the elongated-curved chamber volume and from the elongated-curved chamber volume to a first outlet to transport a first media from the first inlet to the first outlet through a first region of the elongated-curved chamber volume and (ii) a second fluidic channel having smooth flow paths from a second inlet to the elongated-curved chamber volume and from the elongated-curved chamber volume to a second outlet to transport a second media from the second inlet to the second outlet through a second region of the elongated-curved chamber volume, wherein the first internal structure comprises a plurality of fixation elements extending therefrom, wherein the second internal structure comprises corresponding recesses for the fixation elements, wherein the plurality of fixation elements and corresponding recesses provide for fixation of the tissue in the first internal structure and the second internal structure, to provide separation of the first region and the second region.

In some embodiments, the device further includes a first set of electrodes embedded within the first chamber body; and a second set of electrodes embedded within the second chamber body.

In some embodiments, the device further includes a first electronic circuit board having measurement circuitries to couple to the first set of electrodes; and a second electronic circuit board having measurement circuitries to couple to the second set of electrodes, wherein the first electronic circuit board is fixably coupled to the first chamber body, and wherein the second electronic circuit board is fixably coupled to the second chamber body.

In some embodiments, the device further includes a third electronic circuit board having a first interface to the first electronic circuit board, wherein the third electronic circuit board includes a controller to execute a measurement program.

In some embodiments, the first set of electrodes is embedded in a first electrode chip that is operatively coupled to the measurement circuitries of the first electronic circuit, wherein the first electrode chip is positioned within the first region to obtain a first plurality of measurements from a first side of the tissue when inside the device; and wherein the second set of electrodes is embedded in a second electrode chip that is operatively coupled to the measurement circuitries of the second electronic circuit, wherein the second electrode chip is positioned within the second region to obtain a second plurality of measurements from a second side of the tissue when inside the device.

In some embodiments, the first electrode chip and the second electrode chip each comprise at least one voltage electrode and at least one current electrode for obtaining the first plurality of measurements and the second plurality of measurements.

In some embodiments, the first electrode chip and the second electrode chip each comprise a trans-barrier cell electrical resistance sensor (e.g., Trans-Epithelial Electrical Resistance (TEER) sensor) positioned in proximity to a respective side of the tissue sample within the assembled chamber.

In some embodiments, the first electrode chip and the second electrode chip are each configured to measure a trans-barrier cell electrical resistance sensor (e.g., Trans-Epithelial Electrical Resistance) and at least one of pH, oxygen level, glucose level, and lactose level in relation to a respective side of the tissue sample.

In some embodiments, the device is further configured with pairs of pH, O₂, glucose, and/or lactate sensors at each respective side of the tissue sample, e.g., including at least a first side and a second side of the tissue sample, configured to measure metabolites in the media in order to determine a difference in metabolic activity. The pH, O2, Glucose, or lactate sensors, or other sensors described herein, at each side of the chamber/tissue, may be paired to obtain the difference of these metabolites between when the media enters each side of the chamber and when the media exits each side of the chamber. The difference may indicate the level of metabolic activities of the sample tissue inside the chamber. In some instances, the difference information is often more important to one side of the tissue (for example, the mucosal side of gut tissue) than to the other side of the tissue (for example, the serosal side of gut tissue). In some embodiments, the elongated-curved chamber volume comprises a central portion, a front distal portion, a rear distal portion, wherein the central portion is wider than the front and rear distal portions.

In some embodiments, the first inlet and first outlet are positioned at a top position of the first chamber body, wherein the second inlet and second outlet are positioned at a top position of the second chamber body, wherein the first outlet is positioned relatively above the first elongated-curved internal structure of the first chamber body to guide air bubble flow to the first outlet, and wherein the first outlet is positioned relatively above the first elongated-curved internal structure of the first chamber body to guide air bubble flow to the second outlet.

In some embodiments, the plurality of fixation elements are tapered (e.g., conic shape, e.g., spikes).

In some embodiments, The plurality of fixation elements are arranged in a ring or a set of concentric rings.

In some embodiments, the plurality of fixation elements are arranged in a non-uniform pattern.

In some embodiments, the assembled chamber is configured as an Ussing chamber.

In some embodiments, the controller is configured to determine one or more properties of each tissue sample based, at least in part, on a difference between the first plurality of measurements and the second plurality of measurements.

In some embodiments, the controller is configured to perform a data shift operation and/or drift correction on the obtained measurements.

In some embodiments, the controller is operatively coupled to a display or hosting web service for outputting the obtained measurements.

In some embodiments, the first electronic circuit board and the second electronic circuit board each comprise front-end mixed signal acquisition circuitries for the respective set of electrodes.

In some embodiments, the first inlet has an axis that is in-line with that of the first outlet along a first plane, wherein the second inlet has an axis that is in-line with that of the second outlet along a second plane, and wherein the first plane and the second plane are parallel to provide a generally in-line configuration of the first inlet, first outlet, second inlet, and second outlet for the device.

In another aspect, a method is disclosed for operating any one of the above-discussed devices.

In another aspect, a test system is disclosed comprising any one of the above-discussed devices.

Additional advantages will be set forth in part in the description that follows or may be learned by practice. The advantages will be realized and attained by means of the elements and combinations particularly pointed out in the appended claims. It is to be understood that both the foregoing general description and the following detailed description are exemplary and explanatory only and are not restrictive, as claimed.

### BRIEF DESCRIPTION OF THE FIGURES

FIGS. 1A-1C show an example microfluidic chamber for a microphysiological system that can hold explants of live tissue and provide a desired microphysiological environment for the tissue to keep it alive and thriving for an extended period of time for measurements.

FIG. 2A and FIG. 2B are views of an example chamber portion of a device in accordance with certain embodiments of the present disclosure.

FIG. 3A and FIG. 3B are views of an example chamber portion of a device with an integrated electronic circuit board in accordance with certain embodiments of the present disclosure.

FIG. 3C shows an exploded view of a chamber body with an electrode chip in accordance with certain embodiments of the present disclosure.

FIG. 3D shows a fully assembled device in accordance with certain embodiments of the present disclosure.

FIGS. 4A-4C show exploded and detailed views of an example device in accordance with certain embodiments of the present disclosure.

FIGS. 5A-5C show cutaway views and fluid flow paths of an example device in accordance with certain embodiments of the present disclosure.

FIGS. 5D-5H show an example chamber body overlaid with a flow simulation model in accordance with certain embodiments of the present disclosure.

FIG. 6A shows an example electrode chip in accordance with certain embodiments of the present disclosure.

FIGS. 6B-6D are schematic diagrams showing example sensor configurations in accordance with certain embodiments described herein.

FIGS. 7A-7E are schematic diagrams showing an example microphysiological system's supporting electronics in accordance with certain embodiments described herein. FIG. 7D shows an example transepithelial electrical resistance (TEER) acquisition board as an example of a trans-barrier-cell electrical resistance acquisition board in accordance with certain embodiments described herein.

FIG. 8A shows a raw response signal acquired by the example system fabricated in a study.

FIG. 8B is a graph illustrating drift correction performed by the fitting algorithm employed in the example in the study.

FIG. 8C is a graph showing a curve-fitting algorithm applied to flattened raw data.

FIG. 8D is a graph showing a comparison of TEER measured with square wave and 5 kHz sinusoidal stimulus signals.

FIG. 8E is a graph showing the average difference of TEER between sinusoidal and square waveforms for different frequencies of the sinusoidal waveform.

FIG. 8F shows the input level shifter frequency response.

FIG. 8G shows the Howland current source (HCS) frequency response.

FIG. 8H shows the instrumentation amplifier (INA) frequency response.

FIG. 8I shows the Transimpedance amplifier (TIA) frequency response.

FIG. 8J shows the read channel noise power spectral density (PSD) which was found to classify the noise specification of the system.

FIG. 8K shows the step response of the voltage and current stages in the read channel.

FIG. 9A shows results for a control tissue after a 72-hour experiment was conducted.

FIG. 9B shows Collagenase treated, and acidic luminal media resulted in alterations in goblet cell morphology and tight junction expression indicative of increased barrier permeability.

FIG. 9C is a bar graph showing a distinct reduction in TEER after exposure to different media compositions.

FIGS. 9D-9F show results demonstrating physical differences in tissue explant.

Various objects, aspects, features, and advantages of the disclosure will become more apparent and better understood by referring to the detailed description taken in conjunction with the accompanying drawings, in which like reference characters identify corresponding elements throughout. In the drawings, like reference numbers generally indicate identical, functionally similar, and/or structurally similar elements.

### DETAILED DESCRIPTION

To facilitate an understanding of the principles and features of various embodiments of the present disclosure, they are explained hereinafter with reference to their implementation in illustrative embodiments.

In one implementation, a microphysiological system capable of recording the real-time barrier permeability of tissues in a realistic physiological environment over extended durations is provided. Components of the microphysiological system can include a microfluidic chamber designed to hold the live tissue explant and create a sufficient microphysiological environment to maintain tissue viability, proper media composition that preserves a microbiome and creates necessary oxygen gradients across the barrier, integrated sensor electrodes and supporting electronics for acquiring and calculating trans-barrier-cell electrical resistance (e.g., transepithelial electrical resistance (TEER)), and a scalable system architecture to allow multiple chambers running in parallel for increased throughput.

In another implementation, the device (e.g., microfluidic chamber) is further configured with pairs of pH, O₂, glucose, and/or lactate sensors at each respective side of the tissue sample, e.g., at least a first side and a second side of the tissue sample, that are configured to measure metabolites in the media in order to determine a difference in metabolic activity.

### Example Microfluidic Chamber

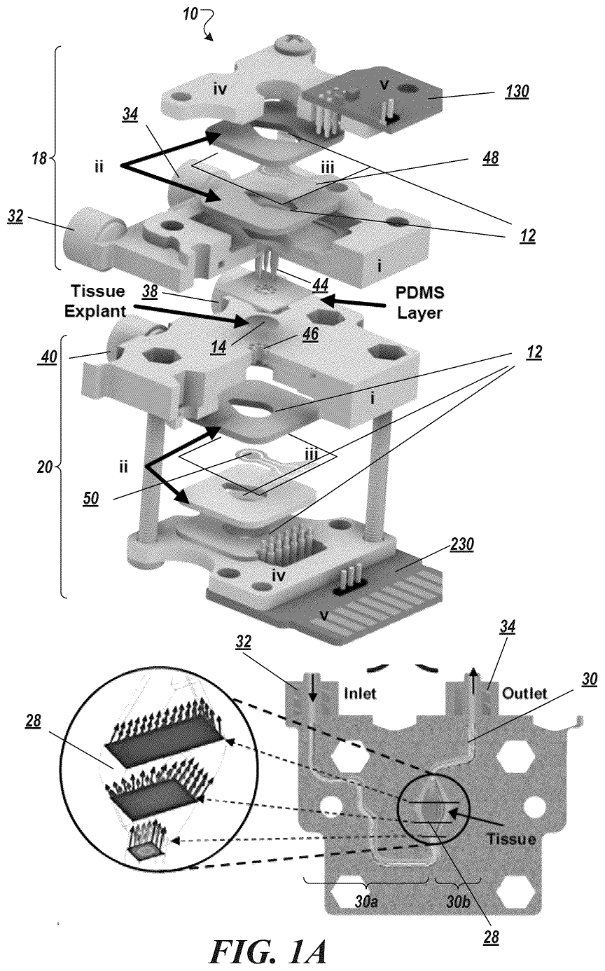

FIGS. 1A-1C show an example microfluidic chamber 12 (also referred to as chamber) for a microphysiological system 10 that can hold explants of live tissue 14 and provide a desired microphysiological environment for the tissue 14 (e.g., barrier cells) to keep it alive and thriving for an extended period of time for measurements. FIGS. 1A and 1B show an exploded cut-view and assembled cut view, respectively, of a microphysiological system 100. FIG. 1A additionally shows the microphysiological system 10 capability in generating laminar flow through the microfluidic chamber with the tissue to provide for the desired microphysiological environment. FIG. 1B additionally shows examples of live tissue explant that could be grown or maintained in the microfluidic chamber. FIG. 1C shows a fully self-contained instrumentation system 16 configured to connect to a number of the microphysiological systems 10 (shown as 10a, 10b, 10c). This disclosure contemplates that different types of tissues can be assessed using the claimed system 10, including tissues for both animal and human subjects, such as, but not limited to, muscle tissue (cardiac muscle tissue, skeletal muscle tissue, smooth muscle tissue, epithelial tissue (skin tissue, organ tissue, reproductive tissue), connective tissue (tendon, bone, fat), endothelial tissue, brain tissue, and/or the like.

In the example shown in FIGS. 1A and 1B, the microphysiological system 10 includes a first chamber body 18 and a second chamber body 20. The first chamber body 18 and the second chamber body 20 have a correspondence to each other such that they can be fixably coupled to one another to form an assembled chamber 22 (see FIG. 1B).

Looking internally at the assembled chamber 22, the first chamber body 18 defines a first elongated-curved internal structure. The second chamber body 20 defines a second elongated-curved internal structure that, together with the first internal structure, defines an elongated-curved chamber volume (e.g., oval or teardrop shape) to hold a tissue sample 14 and to promote laminar flow when media is flowing therethrough.

The first chamber body 18 and the second chamber body 20 each define a first fluidic channel 30 having smooth flow paths (shown as 30a, 30b) from a first inlet 32 to the elongated-curved chamber volume and from the elongated-curved chamber volume to a first outlet 34 to transport the first media from the first inlet 32 to the first outlet 34 through a first region 36 (not shown) of the elongated-curved chamber volume. As shown in the example, the chamber bodies can be made of one or more structures that are fixably attached or subsequently attached or secured to one other.

The first chamber body 18 and the second chamber body 20 also define a second fluidic channel 36 (not shown) having smooth flow paths from a second inlet 38 to the elongated-curved chamber volume and from the elongated-curved chamber volume to a second outlet 40 to transport a second media from the second inlet 38 to the second outlet 40 through a second region 42 of the elongated-curved chamber volume.

The first internal structure comprises a plurality of fixation elements 44 extending therefrom, and the second internal structure 26 comprises corresponding recesses 46 for the fixation elements 44. The plurality of fixation elements 44 and corresponding recesses 46 can provide for fixation of the tissue 14 in the first internal structure and the second internal structure 26, to provide separation of the first region 36 and the second region 42.

The microphysiological system 10 includes a first set of electrodes 48 embedded within the first chamber body 18 and a second set of electrodes 50 embedded within the second chamber body 20.

The microphysiological system 10 further includes a first electronic circuit board 130 having measurement circuitries 54 to couple to the first set of electrodes 48 and a second electronic circuit board 230 having measurement circuitries 58 (not shown) to couple to the second set of electrodes 50. The first electronic circuit board 130 is fixably coupled to the first chamber body 18, and the second electronic circuit board 230 is fixably coupled to the second chamber body 20.

The first set of electrodes 48 is embedded in a first electrode chip 62 that is operatively coupled to the measurement circuitries 54 of the first electronic circuit 130. The first electrode chip 62 is positioned within the first region of the chamber 22, to obtain a first plurality of measurements from a first side of the tissue 14. The second set of electrodes 50 is embedded in a second electrode chip 64 that is operatively coupled to the measurement circuitries 58 of the second electronic circuit 230. The second electrode chip 50 is positioned within the second region of the chamber 22, to obtain a second plurality of measurements from a second side of the tissue 14.

In FIG. 1C, the microphysiological system 10 (shown as 10′) includes a third electronic circuit board 58, or set thereof, having a first interface 60 to the first electronic circuit board 130. The third electronic circuit board 58, or set thereof, includes a controller 60 to execute a measurement program. In the example shown, the third electronic circuit board 58 includes an interface card and a controller card.

The microphysiological system 10 may be manufactured of resin or thermoplastic, e.g., Anycubics UV sensitive resin, using additive manufacturing operation, e.g., Stercolithography (SLA) 3D printer. For UV sensitive resins, to avoid harmful effects from uncured resin, each chamber may be fully cured using UV light and thoroughly rinsed with isopropyl alcohol. The chamber is further sterilized in a low-temperature autoclave. PDMS can be used as a seal between the resin components.

In the example shown in FIGS. 1A and 1B, tubing is connected to each inlet and outlet of the chamber half via a Luer lock connector. Media (e.g., fluid containing nutrients) is then pumped into the chamber using a syringe pump having a controller that can regulate the start and stop time of the flow. With the tissue positioned between the chamber halves, a barrier is formed between the two media flows. The microfluidic path can flow over the opening on each side, exposing the tissue to the media composition. Balanced flow over the tissue inside the chamber can help control the shear stress on the tissue and extend tissue viability.

The chamber was designed using 3D fluid simulations (CFD, Autodesk Inc) to make the media flow over the tissue area with uniform velocity.

Each chamber half has its own PCB breakout board that connects to the glass chip electrodes through gold spring headers. The spring headers are compressed against the chip during assembly. The PCB on the top half chamber has external wire connectors for connecting to the bottom halve PCB. The bottom PCB includes a card edge connector that is plugged into the top of the enclosure for the microphysiological system (see Section System Overview below). The chamber, when plugged into the system, is oriented vertically, making the media flow in from the bottom and out above the tissue. This orientation helps push air bubbles to the top and get them pushed out of the media outlet during experiments. Air bubbles can injure the tissue and cause large deviations from the TEER measurement.

The entire electronic support system is housed in a metal enclosure (FIGS. 1A-C, panels g and h) to shield all electronics from the external physical environment as well as EMF noise caused by electromagnetic radiation. USB ports provide user configuration and control of the experiment from the host computer. The connectors on the microfluidic chambers and the system enclosure are all universal, allowing for plug-and-play functionality. The current implementation can hold up to three chambers at a time. The entire system has a footprint of a typical laptop computer designed to allow it to fit into a limited environment chamber space during experiments. The supporting electronics are responsible for signal acquisition, signal conditioning, and amplification, analog to digital conversion, and communication with the host computer via the USB protocol. A custom-built graphic user interface (GUI) was designed to allow user configurations and real-time control for the experiment. The TEER measurement data are acquired by the host computer, and TEER results are calculated and displayed in the GUI.

In some implementations, the microfluidic chamber 12 is further configured with pairs of pH, O₂, glucose, lactate sensors, and/or other sensors described herein (not shown), at each respective side of the tissue sample (e.g., a first side and a second side of the tissue sample) to determine the difference of the metabolites. For example, a first pair or set of sensors can obtain one or more first measurements when a nutrient flow enters a first side of the chamber 12, and a second pair or set of sensors can obtain one or more second measurements when the nutrient flow exits the first side and/or enters a second side of the chamber 12, where the difference between the one or more first measurements and the one or more second measurements indicates the level of metabolic activities of the sample tissue positioned within the chamber 12.

In some embodiments, the sensors include at least one of a pH sensor, a temperature sensor, a dissolved oxygen sensor, a CO₂ concentration sensor, a salinity sensor, a humidity sensor, a pressure sensor, an ammonia sensor, a sugar sensor (e.g., glucose sensor, fructose sensor, lactate sensor), an amino acid sensor (e.g., glutamine sensor, glutamate sensor), a nucleic acid sensor, a nutrient sensor, or a combination thereof. Examples of the sensor can be an electrochemical sensor or electrode that is configured as a pH sensor, a temperature sensor, a dissolved oxygen sensor, a CO₂ concentration sensor, a salinity sensor, a humidity sensor, a pressure sensor, an ammonia sensor, a sugar sensor (e.g., glucose sensor, fructose sensor, lactate sensor), an amino acid sensor (e.g., glutamine sensor, glutamate sensor), a nucleic acid sensor, a nutrient sensor, or a combination thereof. The sensors may further include surface coatings to enhance selectivity to various analytes, e.g., solid-state electrolytes such as Nafion and/or membrane for enhanced sensitivity to oxygen; glucose oxidase enzyme (GOx) and Nafion for enhanced sensitivity to glucose; and lactose oxidase (LOx) and Nafian for enhanced sensitivity to lactose; among other enzymes.

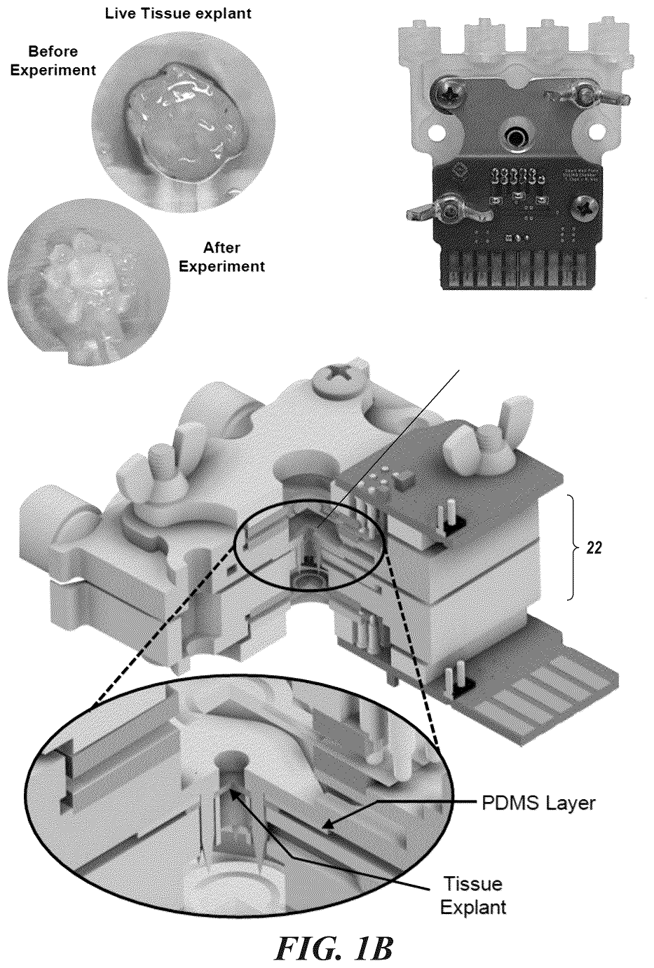

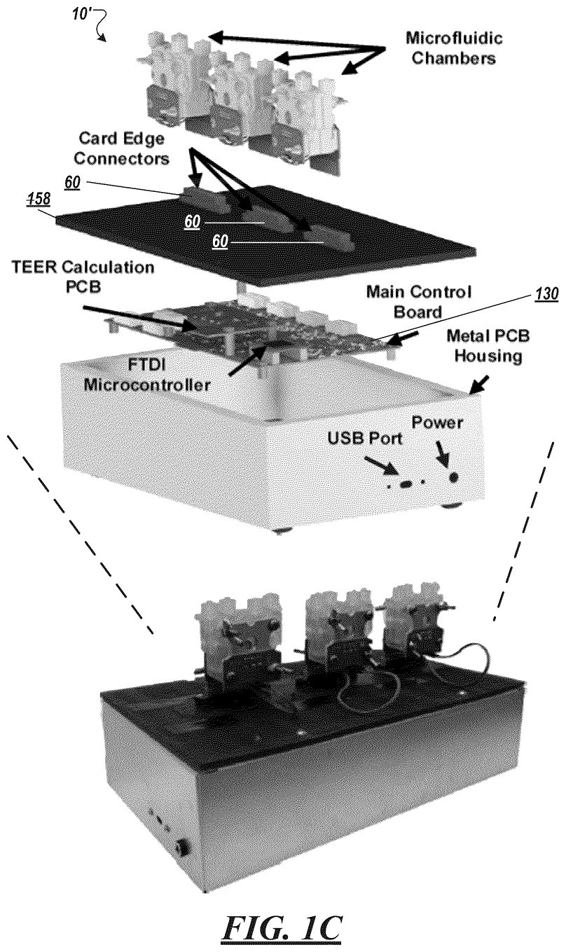

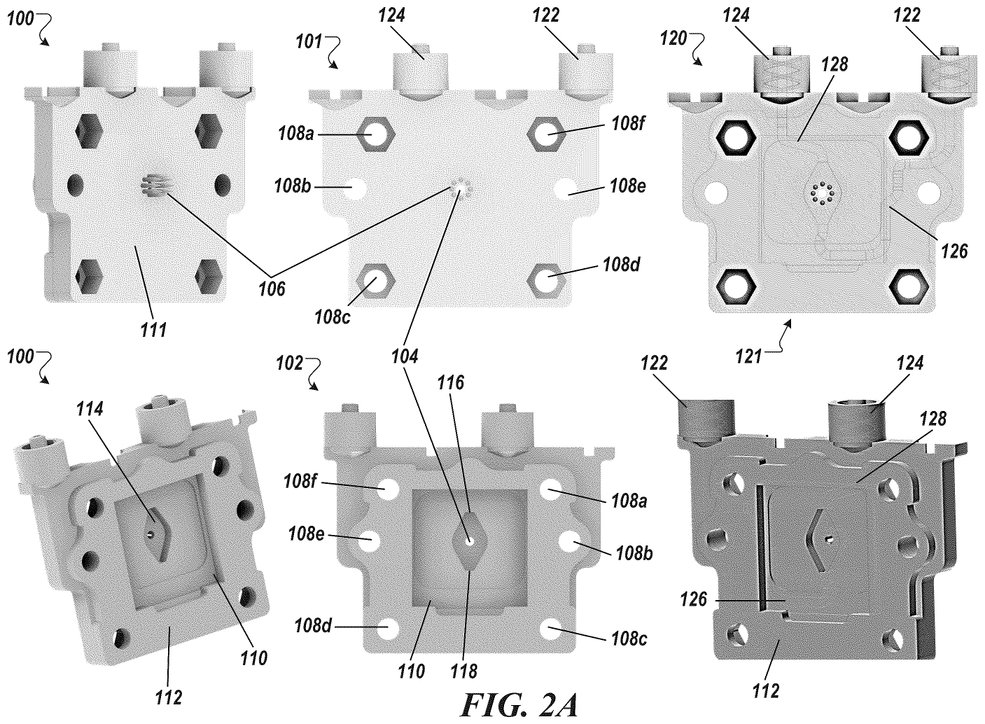

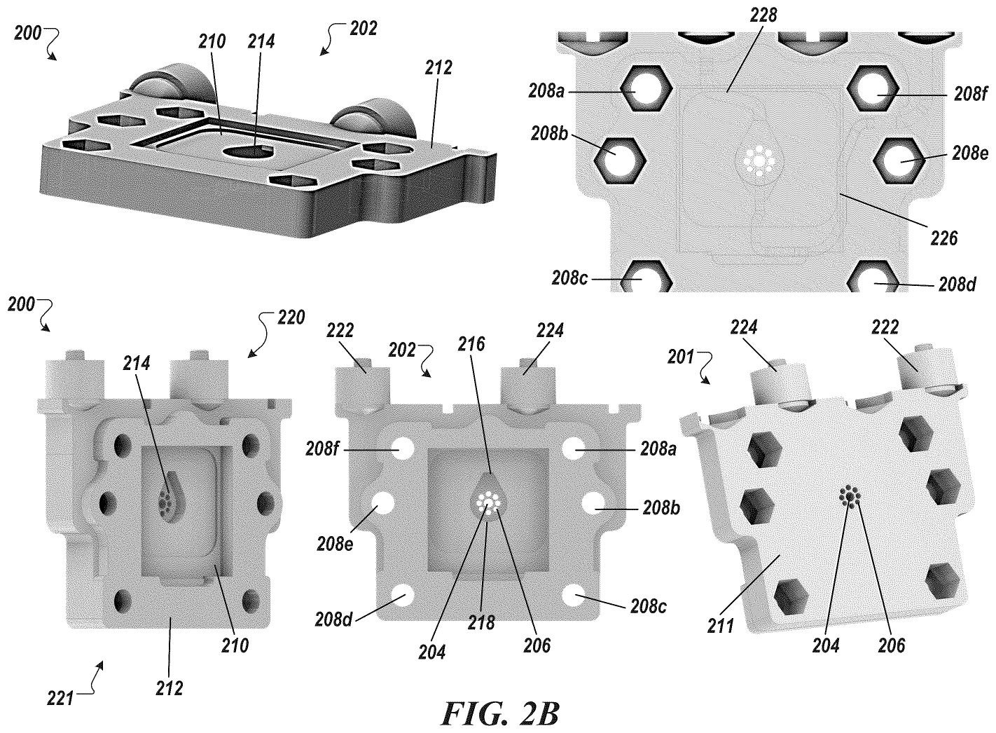

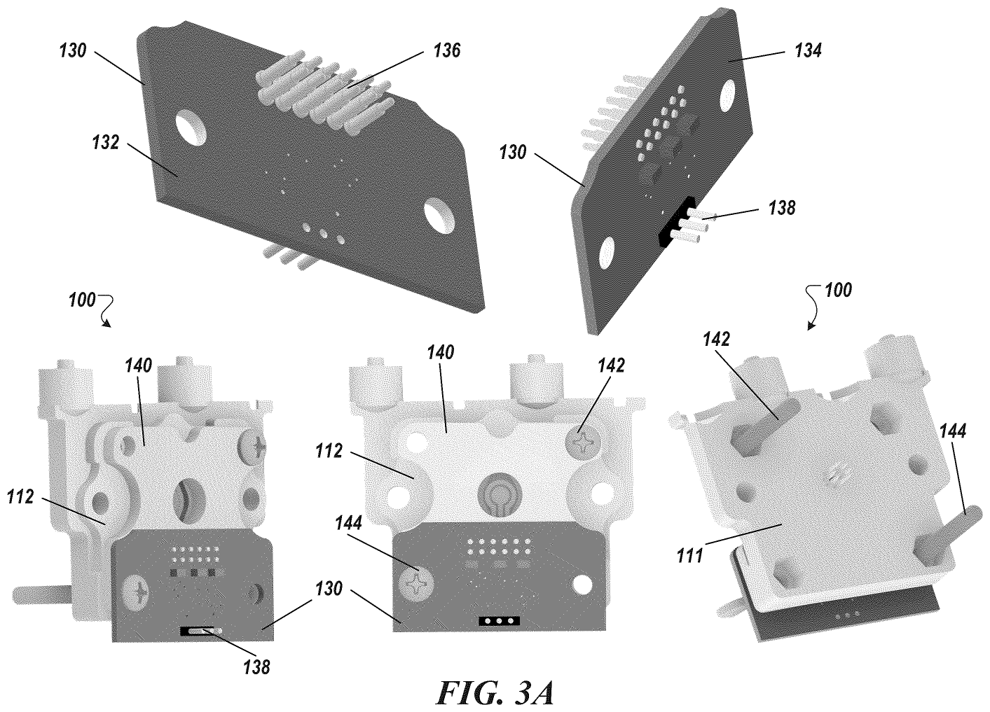

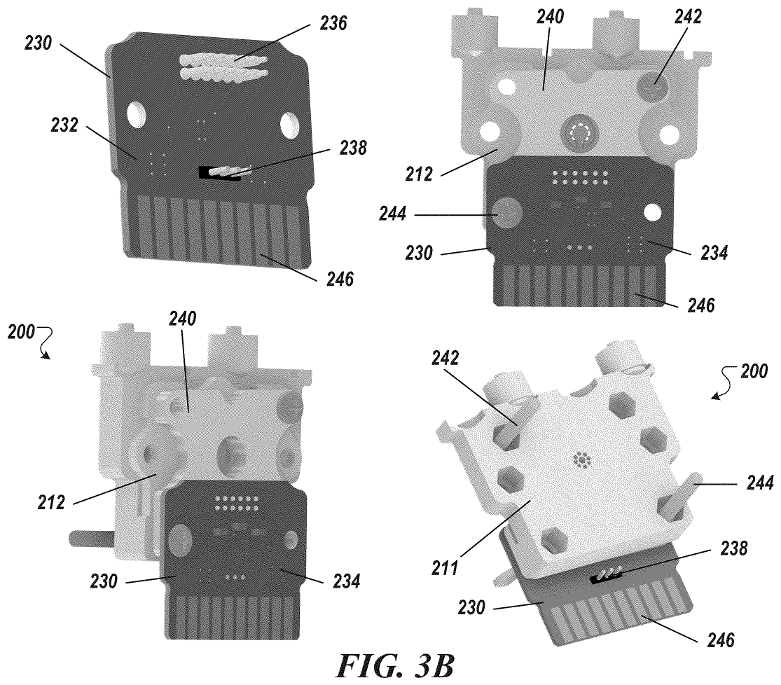

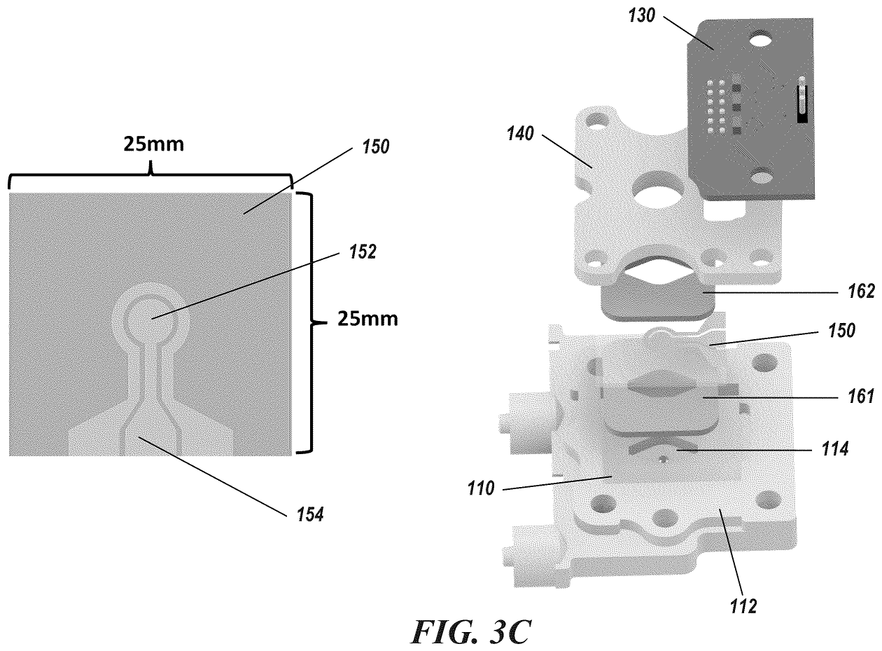

FIGS. 2A-5H show an example device 10 (for example, the microfluidic chamber of FIGS. 1A-C), according to one illustrative implementation. Specifically, FIG. 2A shows the first chamber body 18 (shown as 100) for the first side of the device 10; FIG. 2B shows the second chamber body 20 (shown as 200) for the second side of the device 10; FIG. 3A shows the first chamber body 100 with the associated first printed circuit board (PCB) 130; FIG. 3B shows the second chamber body 200 with the associated second printed circuit board (PCB) 230; FIG. 3C shows an exploded view of the various elements of the second side of the device 10; and FIG. 3D shows the fully assembled device 10. FIGS. 4A-4C show various exploded and detailed views of the device 10 to facilitate understanding of the structure and interaction between the two sides. FIGS. 5A-5H show cutaway views and fluid flow paths of the device 10 for each side individually and as a complete assembly device 10. The terms “fluid flow path,” “flow path,” “flow channel,” “fluidic channel,” and “microfluidic channel” are used interchangeably herein.

FIG. 2A shows the first chamber body 100 of the first side of the device 10. The first side may be termed a “luminal” side because of the orientation of the tissue sample, or it may be termed a “spike” side due to the spike element herein described. The first chamber body 100 includes an inner side 101 and an outer side 102 opposite from the inner side 101. The first chamber body 100 defines a central hole 104 extending between the inner side 101 and the outer side 102. The chamber body 100 further defines a plurality of fastener holes 108a, 108b, 108c, 108d, 108c, and 108f. Each of the fastener holes 108a-108f extends through the first chamber body 100 from the inner side 101 to the outer side 102 and is configured to accept a fastener therein (e.g., for pulling the first and second sides of the device 10 together).

The inner side 101 includes a primarily flat surface 111 and is configured to face towards and interact with the second chamber body 200. The fixation elements 44 (shown and now referenced a plurality of spikes 106) extend substantially perpendicularly from the flat surface 111 of the inner side 101. The spikes 106 are arranged to surround the central hole 104. The fastener holes 108a-108f are also shown extending through the flat surface 111 of the inner side 101. As shown in FIG. 2A, the plurality of spikes 106 on the surface of the first chamber body 100 are configured to mate with corresponding apertures on the surface of the second chamber body 200 in order to anchor a tissue within the device 10. Securely anchoring the tissue within the device 10/chamber addresses issues due to living tissue contraction and/or leaks that would otherwise occur. In some implementations, as shown, the plurality of spikes 106 define/form a concentric ring around the live tissue sample. Additionally, in some examples, the plurality of spikes 106 are each offset from one another (i.e., the plurality of spikes 106 may be slightly non-circular and can be a slightly irregular shape such as a variation of an oval, ellipse, or the like.

On the outer side 102, the first chamber body 100 includes an outer surface 112. The fastener holes 108a-108f are shown extending through the outer surface 112 of the outer side 102. The first chamber body 100 further defines a first recess 110, extending the interior to the first chamber body 100 from the outer surface 112. The first recess 110 has a substantially square shape. The first recess 110 is configured to retain a securing plate (e.g., a PDMS plate).

The first chamber body 100 further defines a fluid flow chamber 114 extending the interior to the first chamber body 100 from the first recess 110. The fluid flow chamber 114 has a substantially diamond shape. The fluid flow chamber 114 is in fluid communication with the central hole 104. The fluid flow chamber 114 is oriented such that a top end 116 and a bottom end 118 of the fluid flow chamber 114 are longitudinally aligned with a top side 120 and a bottom side 121 of the first chamber body 100.

The first chamber body 100 also includes a first Luer lock 122 (previously referenced as a first inlet 32) and a second Luer lock 124 (previously referenced as a first outlet 34) on the top side 120 of the first chamber body 100. Each of the first Luer lock 122 and the second Luer lock 124 define fluid inlets and/or outlets. The first chamber body 100 defines an inlet conduit 126 (shown with shadow lines in FIG. 2A) extending between and in fluid communication with the first Luer lock 122 and the bottom end 118 of the fluid flow chamber 114. The first chamber body 100 further defines an outlet conduit 128 extending between and in fluid communication with the second Luer lock 124 and the top end 116 of the fluid flow chamber 114.

FIG. 2B shows the second chamber body 200 of the second side of the device 10. The second side, in some embodiments, may be termed a “serosal” side because of the orientation of the tissue sample, or it may be termed a “receptacle” side due to the openings/receptacles for the spikes from the first side. The second chamber body 200 includes an inner side 201 and an outer side 202 opposite from the inner side 201. The second chamber body 200 defines a central hole 204 extending between the inner side 201 and the outer side 202. The chamber body 100 further defines a plurality of fastener holes 208a, 208b, 208c, 208d, 208e, and 208f. Each of the fastener holes 208a-208f extends through the second chamber body 200 from the inner side 201 to the outer side 202 and is configured to accept a fastener therein (e.g., for pulling the first and second sides of the device 10 together).

The inner side 201 includes a primarily flat surface 211 and is configured to face towards and interact with the first chamber body 100. A plurality of receptacles 206 are defined on the primarily flat surface 211 of the inner side 201. The receptacles 206 are arranged to surround the central hole 204. The fastener holes 208a-208f are also shown extending through the flat surface 211 of the inner side 201.

On the outer side 202, the second chamber body 200 includes an outer surface 212. The fastener holes 208a-208f are shown extending through the outer surface 212 of the outer side 202. The second chamber body 200 further defines a first recess 210 extending the interior to the first chamber body 200 from the outer surface 212. The first recess 210 has a substantially square shape. The first recess 210 is configured to retain a securing plate (e.g., a PDMS plate).

The second chamber body 200 further defines a fluid flow chamber 214, extending the interior to the second chamber body 200 from the first recess 210. The fluid flow chamber 214 has a substantially teardrop-like shape that facilitates the laminar flow of a media over the tissue sample. Said differently, a central portion of the fluid flow chamber 214, which contains the tissue sample, is wider than the distal portions to facilitate laminar flow of the media (e.g., fluid) over the tissue sample. The fluid flow chamber 214 is in fluid communication with the central hole 204. The fluid flow chamber 214 is oriented such that a top end 216 and a bottom end 218 of the fluid flow chamber 214 are longitudinally aligned with a top side 220 and a bottom side 221 of the second chamber body 200.

The second chamber body 200 also includes a first Luer lock 222 and a second Luer lock 224 on the top side 220 of the second chamber body 200. Each of the first Luer lock 222 and the second Luer lock 224 define fluid inlets and/or outlets. The second chamber body 200 defines an inlet conduit 226 (shown with shadow lines in FIG. 2B) extending between and in fluid communication with the first Luer lock 222 and the bottom end 218 of the fluid flow chamber 214. The second chamber body 200 further defines an outlet conduit 228 extending between and in fluid communication with the second Luer lock 224 and the top end 216 of the fluid flow chamber 214.

FIG. 3A shows the first chamber body 100 with the associated first printed circuit board (PCB) 130 and other structural elements. The PCB 130 has an inner surface 132 and an outer surface 134. The PCB 130 includes a plurality of spring headers 136 extending out from the inner surface 132. The spring headers 136 will contact and connect to chip electrodes, as further described below. The PCB 130 also includes a wire connector 138 configured to couple to a wire that extends to the PCB 230 coupled to the second chamber body 200, as shown in FIG. 3D. A compression plate 140 is shown in FIG. 3A coupled to the outer surface 112 of the first chamber body 100. The compression plate 140 retains various elements within the first recess 110, as further described below. The outer surface 134 of the PCB 130 abuts the compression plate 140. A first fastener 142 extends through the fastener hole 108a to hold one portion of the compression plate 140. A second fastener 144 extends through the fastener hole 108d to hold both the PCB 130 and the compression plate 140.

FIG. 3B shows the second chamber body 200 with the associated second printed circuit board (PCB) 230 and other structural elements. The second PCB 230 has an inner surface 232 and an outer surface 234. The PCB 230 includes a plurality of spring headers 236 extending out from the inner surface 232. The spring headers 236 will contact and connect to chip electrodes, as further described below. The PCB 230 also includes a wire connector 238 configured to couple to a wire that extends to the PCB 230 coupled to the first chamber body 100, as shown in FIG. 3D. The second PCB 230 also includes a card edge connector 246 configured to plug into and connect with a microphysiological system.

A compression plate 240 is shown in FIG. 3B coupled to the outer surface 212 of the second chamber body 200. The compression plate 240 retains various elements within the first recess 210, as further described below. The outer surface 234 of the PCB 230 abuts the compression plate 240. A third fastener 242 extends through the fastener hole 208a to hold one portion of the compression plate 240. A fourth fastener 244 extends through the fastener hole 208d to hold both the PCB 230 and the compression plate 240.

FIG. 3C shows an exploded view of the first chamber body 100 with the PCB 130, the compression plate 140, a gold electrode chip 150, and two substrate layers, including a first substrate layer 161 and a second substrate layer 162 (e.g., PDMS layers). FIG. 3C also shows a larger representation of the gold electrode chip 150. The structure and function of the gold electrode ship 150 and the substrate layers 161, 162 with respect to the first chamber body 100 is substantially the same as the gold electrode chip 250 and substrate layers 261, 262 associated with the second chamber body 200.

The first substrate layer 161 is disposed within the first recess 110 of the first chamber body 100. The first substrate layer 161 has a substantially square shape matching the shape of the first recess 110. The first substrate layer 161 includes a central opening matching the diamond-like shape of the fluid flow chamber 114.

The gold electrode chip 150 is disposed on top of the first substrate layer 161. A central portion 152 of the gold electrode chip 150 is centrally aligned with the fluid flow chamber 114 and the central opening of the first substrate layer 161. A second portion 154 of the gold electrode chip 150 extends away from the fluid flow chamber 114 and outside the bounds of the first substrate layer 161. The second portion 154 of the gold electrode chip 150 is configured to contact the plurality of spring headers 136 from the PCB 130 (e.g., to relay information about the fluid flow chamber 114 and associated fluid to the PCB 130 and related systems.

The second substrate layer 162 is disposed on top of the gold electrode chip 150. The compression plate 140 is then installed over the second substrate layer 162 to retain the second substrate layer 162 within the first recess 110. The second substrate layer 162 has a similar shape and central opening as the first substrate layer 161. Together, the first substrate layer 161 and the second substrate layer 162 hold the gold electrode chip 150 in place and facilitate the retention and placement of the gold electrode chip 150.

FIG. 3D shows several views of a fully assembled device 10 with both the first chamber body 100 and the second chamber body 200 coupled together-including images of prototype device examples. In the assembled device 10, the first PCB 130 and the second PCB 230 are coupled together via a wire 139 between the wire connector 138 and the wire connector 238. The flat surface 111 of the first chamber body 100 is shown facing the primarily flat surface 211 of the second chamber body 200 in the assembled state. Furthermore, the first fastener 142, second fastener 144, third fastener 242, and fourth fastener 244 extend through each of the first chamber body 100 and the second chamber body 200 to hold the sides together, as shown.

FIG. 4A and FIG. 4B each shows exploded views of a fully assembled device 10. Importantly, the device 10 of FIG. 4A shows the tissue sample 14 visible in between the first chamber body 100 and the second chamber body 200. A central substrate 14 (e.g., a PDMS layer) is also disposed between the first chamber body 100 and the second chamber body 200.

During assembly, the tissue sample 14 is placed between the first chamber body 100 and the second chamber body 200, along with the central substrate 14. Specifically, the tissue sample 14 and central substrate 14 are placed between the plurality of spikes 106 on the flat surface 111 of the first chamber body 100 and the plurality of receptacles 206 on the flat surface 211 of the second chamber body 200. When the tissue sample 14 is enclosed in the device 10, the plurality of spikes 106 puncture around the outer edge of the tissue sample 14. The center of the tissue sample 14 is left untouched. The central substrate 15 has openings corresponding to the plurality of spikes 106. The central substrate 14 creates a flush seal against the tissue sample 14 to prevent any leaks between the chamber halves on either side of the device.

As shown in more detail in FIG. 4C, the fluid flow chamber 114 of the first chamber body 100 is formed on one side of the tissue sample 14, while the fluid flow chamber 214 of the second chamber body 200 is formed on the opposite side of the tissue sample 14. Therefore, the fluid flowing through the fluid flow chamber 114 (e.g., from the first Luer lock 122 through the inlet conduit 126 into the fluid flow chamber 114 and through the outlet conduit 128 to the second Luer lock 124 out of the first chamber body 100) contacts only one side of the tissue sample 14. Simultaneously, the fluid flowing through the fluid flow chamber 214 (e.g., from the first Luer lock 222 through the inlet conduit 226 into the fluid flow chamber 214 and through the outlet conduit 228 to the second Luer lock 224 out of the first chamber body 100) contacts only the opposite side of the tissue sample 14.

FIGS. 5A-5H show additional details of the above-described flow path. For example, FIG. 5A shows a cross-section of the first chamber body 100. FIG. 5A more clearly shows the fluid flow path from the first Luer lock 122, through the inlet conduit 126, through the fluid flow chamber 114, through the outlet conduit 128, and exiting via the second Luer lock 124.

FIG. 5B also shows the cross-section of the first chamber body 100 with example sensors placed in the fluid flow chamber 114. As shown, at least two metabolic sensors and at least one transepithelial electrical resistance (TEER) sensor (as an example of a trans-barrier cell electrical resistance sensor) are present in the fluid flow chamber 114.

FIG. 5C shows the second chamber body 200 with various cross-sectional and sectioned views. FIG. 5C more clearly shows the fluid flow path from the first Luer lock 222, through the inlet conduit 226, through the fluid flow chamber 214, through the outlet conduit 228, and existing via the second Luer lock 224.

FIG. 5D shows the first chamber body 100 with a flow simulation model illustrated in the fluid flow conduits 126, 128 and the fluid flow chamber 114. Similarly, FIG. 5E shows the first chamber body 100 cross-section with the sensors in the fluid flow chamber 114. FIGS. 5F and 5G similarly show the second chamber body 200 with a fluid flow simulation illustrated in the flow conduits 126, 128 and the fluid flow chamber 214. As shown, the flow through the fluid flow chamber 114 and the fluid flow chamber 214 is largely laminar flow, simulating that of an organic body. As depicted in FIG. 5D, in some embodiments, a central portion of the fluid flow chamber containing the tissue sample is wider than the distal portions to facilitate the laminar flow of the media over the tissue sample.

FIG. 5H shows the assembled device 10 with each flow path simulated through the first chamber body 100 and the second chamber body 200. Each fluid flow from each side of the device 10 approaches each other in the corresponding fluid flow chamber 114 and fluid flow chamber 214 on either side of the tissue sample 14. For example, a first fluid may flow through the fluid flow chamber 114 of the first chamber body 100 on one side of the tissue sample 14, while a second fluid may flow through the fluid flow chamber 214 of the second chamber body 200 on the opposite side of the tissue sample 14. The sensors on the gold electrode chip 150 and the gold electrode chip 250 then relay information about the interaction between either side for the tissue sample 14.

### Example Measurement System

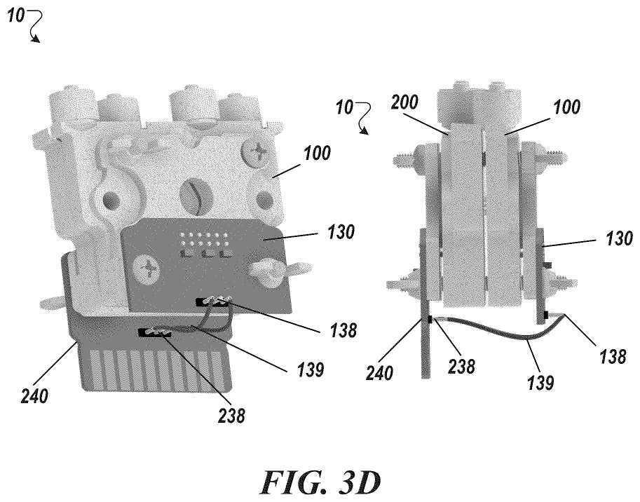

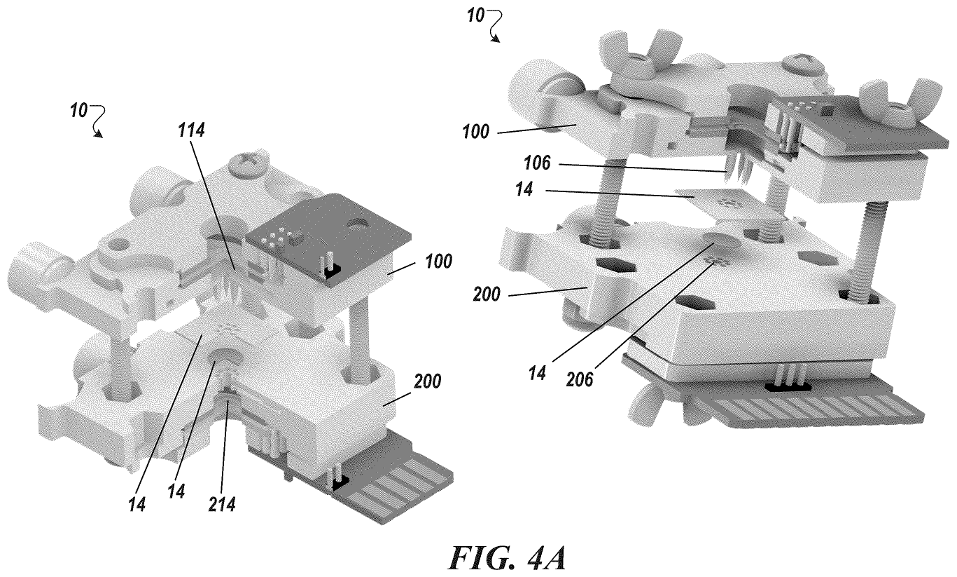

FIG. 6A-6D are schematic diagrams showing example sensor configurations (e.g., electrode chip or sensing component comprising TEER and metabolic sensors) within an example chamber in accordance with certain embodiments described herein.

FIG. 6A shows an example electrode chip 250, including a first metabolic sensor 602, a second metabolic sensor 604, and a TEER sensor 606. A media 605 flows through the tissue 601, which is shown positioned near the location of the TEER sensor 606. The metabolic sensors 602, 604 measure target metabolite(s) between and after the media 605 is exposed to the tissue 601 to derive the metabolite consumption/production rates.

FIG. 6B, FIG. 6C, and FIG. 6D each shows exploded views of an example device 10. FIG. 6A and FIG. 6B show the assembled chamber body 115 (comprising the first chamber body 100 and second chamber body). In FIG. 6D, the tissue sample 14 is visible in between the first chamber body 100 and the second chamber body 200. A central substrate 15 (e.g., a PDMS layer) is also disposed between the first chamber body 100 and the second chamber body 200. As discussed above, during assembly the tissue sample 12 is placed between the first chamber body 100 and the second chamber body 200, along with the central substrate 15. The device 10 includes a first electrode chip 150 and a second electrode chip 250 that can each comprise an arrangement of TEER and metabolic sensors, such as, but not limited to, the configuration described in connection with FIG. 6A.

In various implementations, the device 10 includes current injector instrumentation that injects a current into the device 10. To avoid intrinsic error on the impedance measurement, the device 10 is configured to facilitate four-point measurement via the electrode chips 150, 250. A two-point setup introduces unwanted lead resistance from the electrodes to the sample impedance.

For example, two current measurements and two voltage measurements can be obtained with respect to the tissue 14. A first current measurement and a first voltage measurement can be obtained via the first electrode chip 150 on a first side of the tissue 14 within a first chamber portion, and a second current measurement and a second voltage measurement can be obtained via the second electrode chip 250 on a second side of the tissue 14 within the second chamber portion. Using a four-point setup, the voltage electrodes read the voltage directly across the tissue sample 14, separate from the current electrodes. This avoids any potential drop from the current lead resistance.

In some implementations, the first electrode chip obtains tissue measurements before a media flows over the tissue, and the second electrode chip 250 obtains tissue measurements after the media flows over the tissue from a device inlet to a device outlet in a laminar fashion. As discussed above, the shape of the chamber is configured to produce and maintain a laminar flow (e.g., 80%-90% laminar flow) over the tissue. In some implementations, the first electrode chip 150 obtains measurements from a first side of the tissue and the second electrode chip 250 obtains measurements from a second side of the tissue (i.e., opposing sides of the tissue sample). The first and second electrode chip 150, 250 can be used to measure concentrations of various metabolic substances (e.g., oxygen consumption).

Air Bubble Elimination: As the media 605 flows through the device 10 and in response to the injected current, air bubbles may form, which can skew or negatively impact measurement accuracy. As described in more detail above, the assembled chamber body 1115 comprises one or more fluidic channels that begin at a first location (e.g., inlet ending at first Luer lock 122) and terminate at a second location (e.g., outlet ending at second Luer lock 124). The assembled device 10 is configured to be oriented vertically (FIG. 2A), with the device inlet and outlet located parallel to one another and positioned on a top surface of the device 10 (i.e., above the assembled chamber 115). As a result of this inlet/outlet orientation, gravitational forces acting on the device 10 during use will pull any air bubbles to a top portion of the device 10 where they will not affect tissue measurements being obtained in a central portion of the device 10.

### Experimental Results and Additional Examples

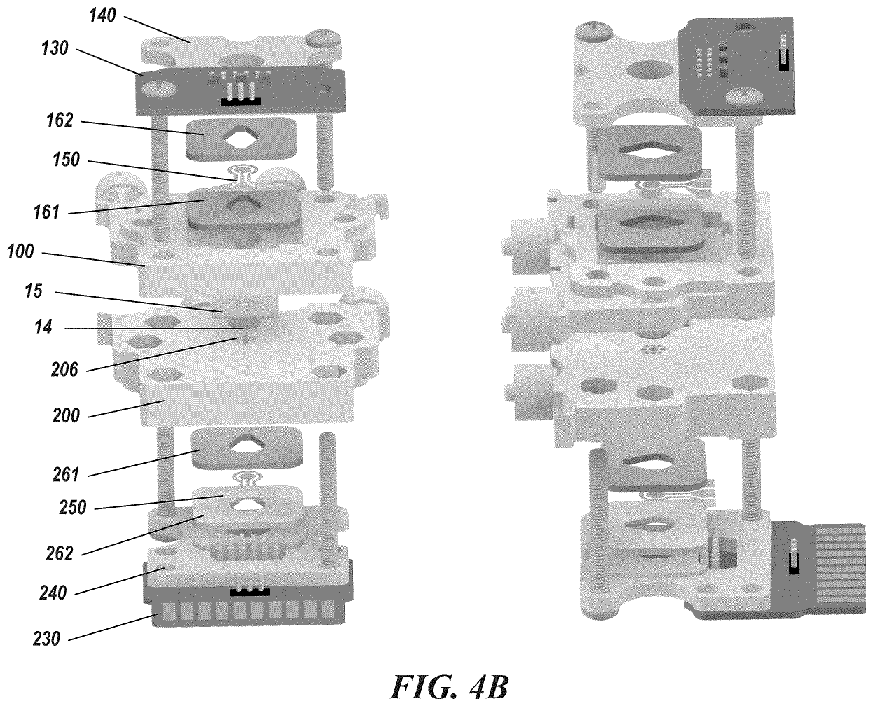

A study was conducted to evaluate the exemplary systems and methods described herein. FIG. 7A-7E are schematic diagrams showing an example microphysiological system's supporting electronics in accordance with certain embodiments described herein.

Electronic Circuits for TEER Measurement or Trans-Barrier-Cell Electrical Resistance: FIG. 7A is a block diagram of example electronic circuitry for performing TEER measurement, as an example of trans-barrier-cell electrical resistance, that shows the flow of signal conditioning and processing in accordance with certain embodiments of the present disclosure. The TEER measurement in the study is operated in the constant-current mode to avoid accidental over-current to damage electrodes and tissues inside the chamber. In the example shown in FIG. 7A, an FTDI microcontroller 702 is used to provide an interface between a host computer 701 and the on-board electronics 704 using a USB protocol 705. This interface allows users to control all internal enable signals from the host computer 701 GUI. A signal generator 703 is used to generate a user-defined AC voltage signal 710. This voltage signal 710 is used to control a Howland current source (HCS) 712 (as shown 712a, 712b, 712c), creating an AC current stimulus signal to the TEER electrodes 734.

Referring now to FIG. 7B, a read channel circuit responsible for supplying stimulus signal and reading output voltage and current signals directly from the sensor electrodes inside the chamber is shown. The HCS 712 is chosen because it can easily achieve high output impedance, signal to noise ratio (SNR), and is fully programmable through external control voltages [23,24]. Up to three parallel current stimulation signals are used for three independent chambers (714a, 714b, 714c in FIG. 7A) in the system. The read-channel (FIG. 7B) consists of a transimpedance amplifier to convert the input current signal (Iᵢₙ) to an output voltage (Vₒᵤₜ), and an instrumentation amplifier to acquire the voltage response from the tissue barrier. A relay 720 is placed in parallel with each microfluidic chamber 714 to discharge built-up charge on the TEER electrodes 734 when necessary. Built-up charge on the electrodes 734 is capable of altering the DC voltage at the input to the microfluidic chamber 714, and, therefore, has a large enough effect to shift the DC voltage towards supply rails, reducing the dynamic range of TEER measurement.

The HCS 712 shown in FIG. 7B uses an ultra-low offset voltage operation amplifier 722 and consists of five high-precision film resistors and a single feedback capacitor for bandwidth control. High output impedance is achieved by resistor matching. The response TEER voltage from the chamber 714 is read by an instrumentation amplifier (INA) 724 with a low input bias current. The INA gain is controlled by the resistor Rg. The response TEER current from the chamber 714 is read using a transimpedance amplifier 726 (TIA), with the current gain set by R_{gain}. An analog-to-digital converter (ADC) 706 is used to convert the amplified analog outputs from the read channel to a digital signal that is sent to the host computer 701 via USB port 705. Due to the different operating voltages of different components along the signal chain to achieve the required output dynamic range, electronic level shifting is required at various points in the system. They are performed by operational amplifiers where the voltage gain is controlled by onboard resistors, and DC shift is adjusted using a digital potentiometer. The digital potentiometer is calibrated before each measurement using a binary search algorithm to find the smallest DC offset current. The input stimulus was also designed to have a high and low current setting to maximize the system's output dynamic range.

FIG. 7D shows an example TEER acquisition board 750, as an example of a trans-barrier cell acquisition board, containing all read channels, digital potentiometers, connectors for the electrodes, and connectors to the main control board 700. The supporting electronics are partitioned into two separate PCB boards. The main control board 700 shown in FIG. 7E contains the external power supply 704, USB connectors, microcontroller 702, ADC 706, signal generators 703, level shifters for control signals, and connectors to the TEER acquisition board. The TEER acquisition board 750 depicted in FIG. 7D contains the HCSs (e.g., 712a, 712b, 712c), the read channels for each chamber (716a, 716b, 716c), the calibration digital potentiometers 717, and the connections for the card edge connectors (718a, 718b, 718c). Even though the system described in connection with FIG. 7A-7E allows three chambers (714a, 714b, 714c) to be used at a time, the system architecture was designed to be scalable to allow expansion to accommodate more chambers.

Electrode Design and Manufacturing: FIG. 7C shows an example TEER electrode chip 734 in accordance with certain embodiments described herein. The electrode chip 734 is manufactured in gold on a glass substrate 736. Each electrode chip 734 consists of two gold (Au) electrodes to allow 4-point measurement. The electrode chip 734 shown in FIG. 7C was fabricated on a 25 mm×25 mm glass substrate through an in-house photolithography, deposition, and lift-off process. The mask was designed using AutoCAD software (Autodesk, Inc.) and manufactured by Artnet Pro (San Jose, CA). The full photolithography steps are described in [25]. In the example shown in FIG. 7C, the outer ring electrode is the current electrode 734a, and the middle circle is the voltage electrode 734b.

Chamber Sterilization: To prevent infection during experiments, all components of the microfluidic chamber (e.g., 714, including chamber body, glass electrode chip, PDMS layers, PCBs, tubing, and Luer locks) were put through the first round of sterilization protocol: (1) 20-minute bath in 1:10 bleach to water ratio, (2) 10-minute soapy water bath inside of ultrasonic cleaner, (3) Next, thoroughly rinse with DI water, (4) 45-minute bath in 70% ethanol, and (5) Finally, thoroughly rinse with deionized (DI) water and let air dry. After the first round of sterilization, the chamber was fully assembled with metal screws and clamps that had been autoclaved (30-minute gravity cycle). After the chambers were assembled, the chambers went through low-temperature gas sterilization and were kept in a sealed bag before use.

Animals, Tissue Collection, and Media Preparation: In all experiments, male C57BL/6 background mice aged 3-4 months were used. Mice were kept on a 12-h light/dark cycle with access to standard chow and water ad libitum. Animal protocols were approved by the Institutional Animal Care and Use Committee (IACUC) at Colorado State University under United States Department of Agriculture (USDA) guidelines. Mice were deeply anesthetized with isoflurane and terminated via decapitation to prepare for tissue collection. The intestines were removed and immediately placed in 4° C. 1× Krebs buffer (in mM: 2.5 KCl, 2.5 CaCl₂, 126 NaCl, 1.2 MgCl₂, 1.2 NaH₂PO₄). To prevent contractions during dissection, the Krebs buffer contained 1 μl/1 mL 1 mM nicardipine (Sigma Aldrich, St. Louis, MO), an L-type calcium ion channel blocker. The colon was then dissected to remove any remaining mesentery. For experiments in which muscle was removed, a 26 G needle was used to gently tease away the muscle layer on the mesenteric edge of the tissue. Tissue was then cut longitudinally using angled vascular scissors to form flat pieces of tissue around ˜5 mm. The study contemplated that other cells, tissues, and barrier cell tissue, among others, as described herein, may be measured using the system of the study.

Adult Neurobasal media was custom-made in-house with 2% B27 supplement (Thermo Fisher scientific, Waltham, WA), 4 mM glucose, 3% 1 M HEPES buffer (Sigma Aldrich, St. Louis, MO), without phenol red. To help maintain the gut microbiome, luminal media contained 0.4 mg/ml inulin (soluble fiber) and 0.5 M sodium sulfite (oxygen scavenger) to decrease oxygen levels [17]. The serosal media had ambient levels of oxygen, creating an oxygen gradient across the tissue, which was previously demonstrated to be necessary for the preservation of a physiologically relevant bacterial community. After 24 h, luminal media for control tissue was not changed. The treatment group luminal media contained 5.80*10-2 U of broad spectrum bacterially sourced collagenase (Worthington Biochemical, Lakewood, NJ) or was treated with hydrochloric acid (HCl) to acidify the pH to 2. After completion of experiments, 0.05 M phosphate-buffered saline (PBS) containing 0.5% cetylpyridinium chloride (CPC) was gently pipetted onto the tissue to preserve the mucus layer. The tissue was then gently removed from the device and placed in 4% paraformaldehyde (PFA) containing 0.5% CPC at 4° C. for 24 h. The tissue was stored in PBS at 4° C. until sectioning.

Tissue Sectioning and Histochemistry: Detailed methodology can be found in our previous publication [11]. Briefly, 1-3 mm sections of the colon were submerged in agarose until polymerization. Tissue was then cut on a vibrating microtome (VT100S; Leica microsystems, Wetzlar, Germany) at a thickness of 50 μm. For lectin and immunohistochemistry, sections were first washed in 1×PBS, then incubated in 0.1M glycine followed by PBS washes and incubated in 0.5% sodium borohydride followed by PBS washes. Sections were then blocked in PBS with 5% normal goat serum (NGS; Lampire Biological, Pipersville, PA), 1% hydrogen peroxide, and 0.3% Triton X (TX). Next, sections were placed in PBS containing 0.3% TX and 5% NGS with the appropriate lectin or antibody for 2 days. The lectin used was Ulex Europaeus Agglutinin I conjugated to Rhodamine (UEA-1; Vector Labs) at a concentration of 0.125 μg/mL. Primary antibodies used were anti-claudin1 (Invitrogen) 1:200 and anti-peripherin (Sigma-Aldrich) 1:300. After lectin or primary antibody incubation, sections were washed in PBS with 1% NGS. Sections incubated in primary antibodies were then incubated with PBS containing 0.02% TX and Alexa Fluor 594 conjugated to secondary antibodies specific to the species of the primary antibodies at a 1:500 dilution. Finally, sections were washed in PBS, mounted on slides, and the cover slipped. Images were taken using a Zeiss LSM800 upright confocal laser scanning microscope and a 20× (W Plan-Apochromat 20×/1.0 DIC Vis-ir ∞/0.17) objective or an Olympus BH2 brightfield microscope.

TEER Calculation: Processing of TEER signals involves conditioning steps to reduce noise and other artifacts. The conditioned signals were further processed by applying a curve-fitting algorithm to obtain the magnitude and phase of the voltage and current response signals. The impedance magnitude (|Z|) and phase difference (θ_{diff}) can then be calculated using Eqs. (1) and (2). Where Avcurrent and Avvoltage are the current and voltage gain values, respectively.

$$\vert Z \vert = \frac{{V}_{peak}}{{I}_{peak}} * \frac{{Av}_{current}}{{Av}_{voltage}} \qquad ( 1 )$$

$${θ}_{diff} = {θ}_{voltage} ( deg ) - {θ}_{current} ( deg ) \qquad ( 2 )$$

The magnitude and phase values are determined for each frequency to obtain the impedance spectrum of the tissue sample, commonly referred to as electrical impedance spectroscopy (EIS). Due to its versatility of revealing impedance information across a wide range of frequencies, EIS is a widely-used technique to discover the impedance characteristics of tissue/cell-culture samples in Ussing Chambers, Organ-on-a-chip devices, and well inserts [8], [12], [19], [20], [23], [26]-[31].

Similar condition steps may be applied to barrier-cell electrical resistance signals.

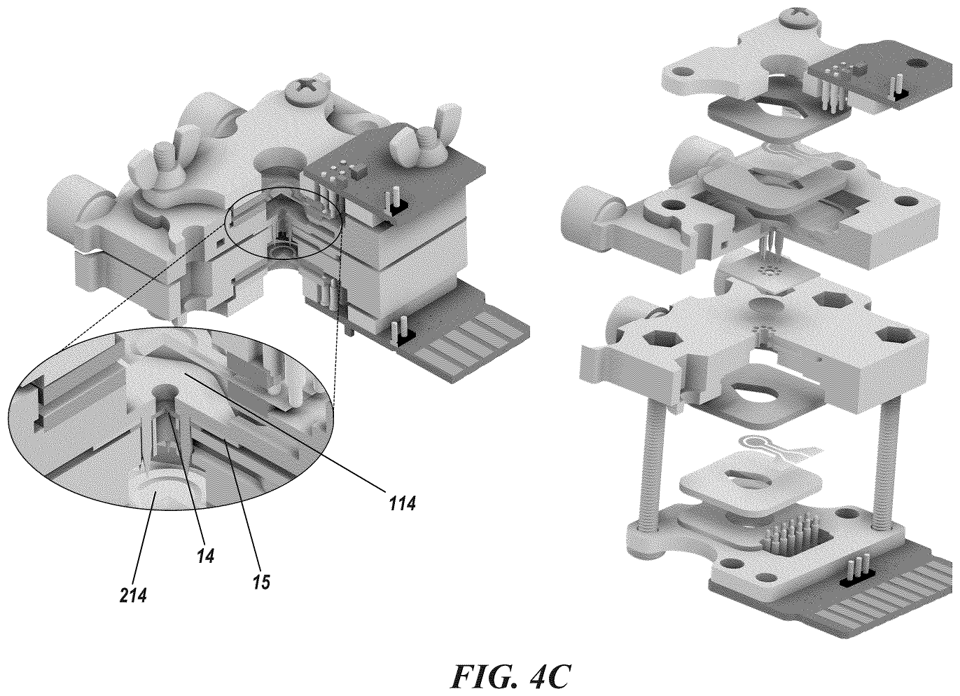

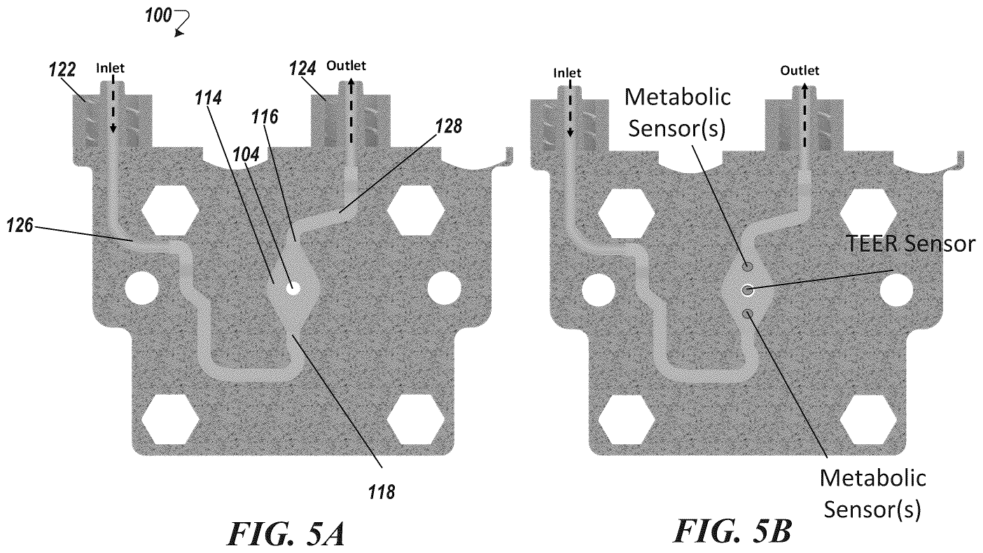

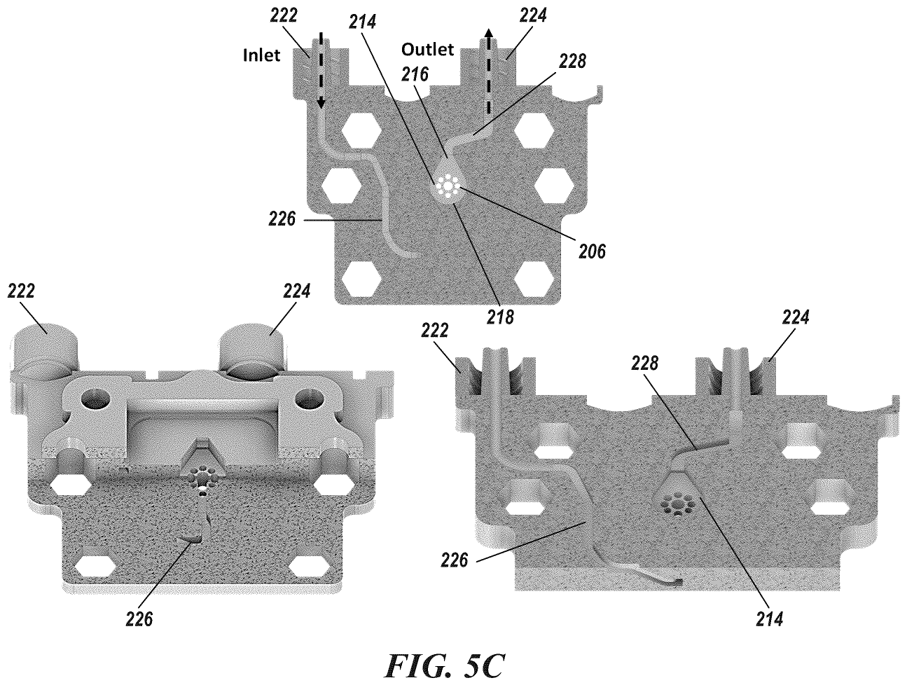

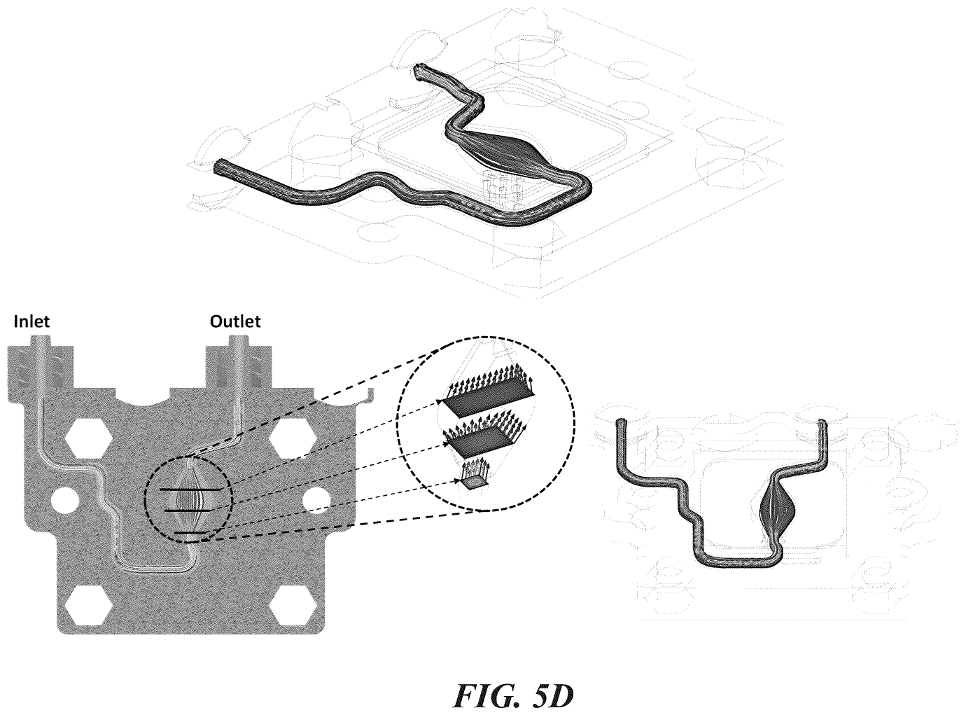

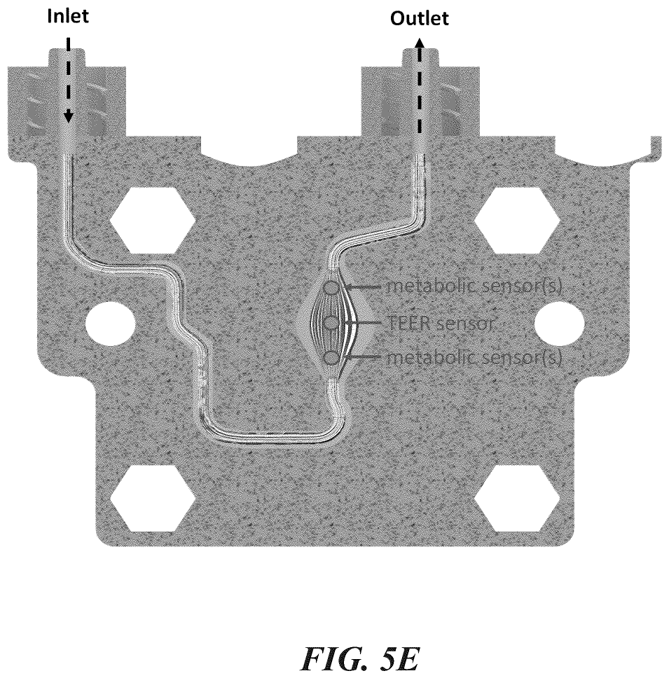

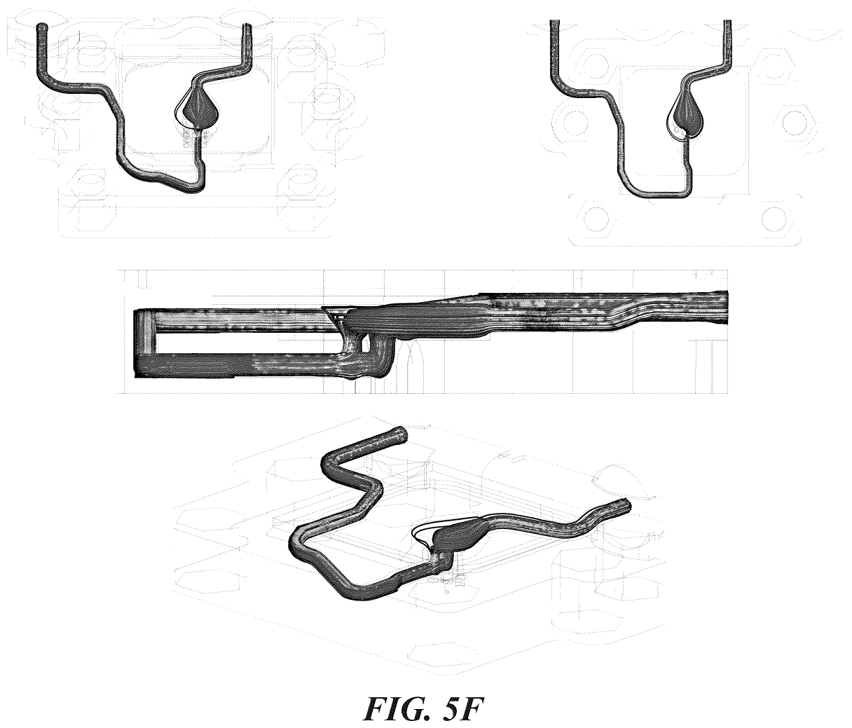

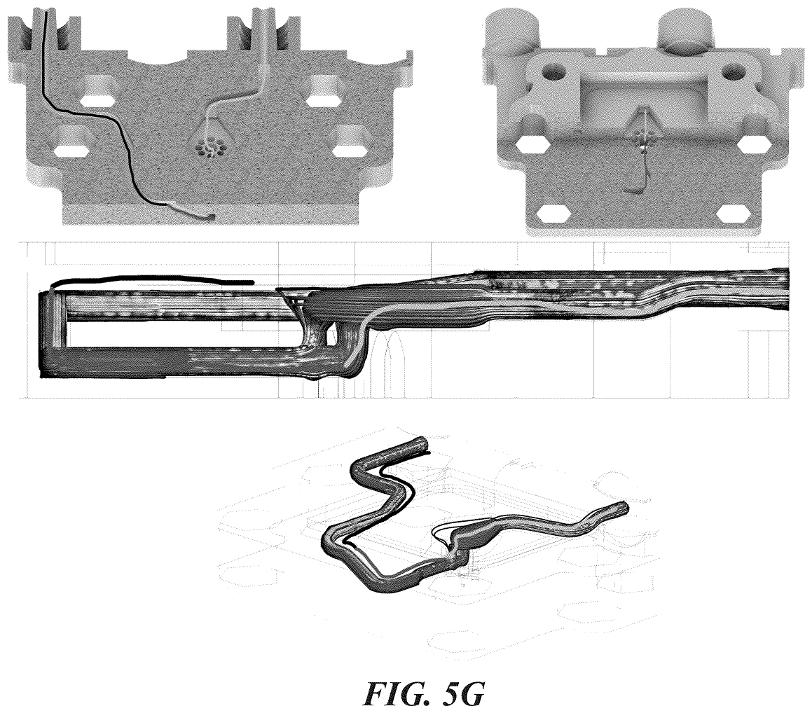

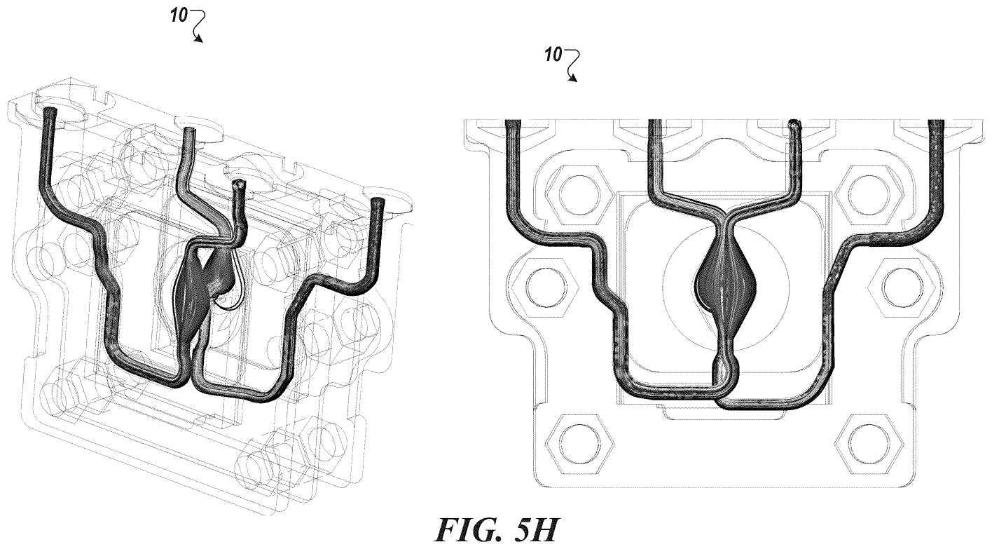

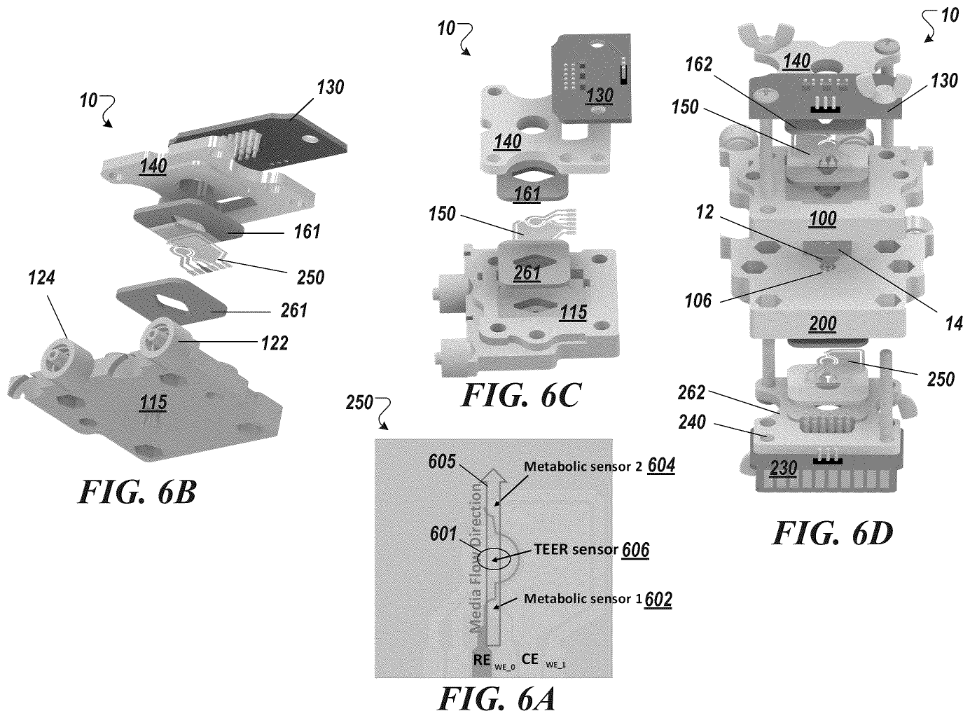

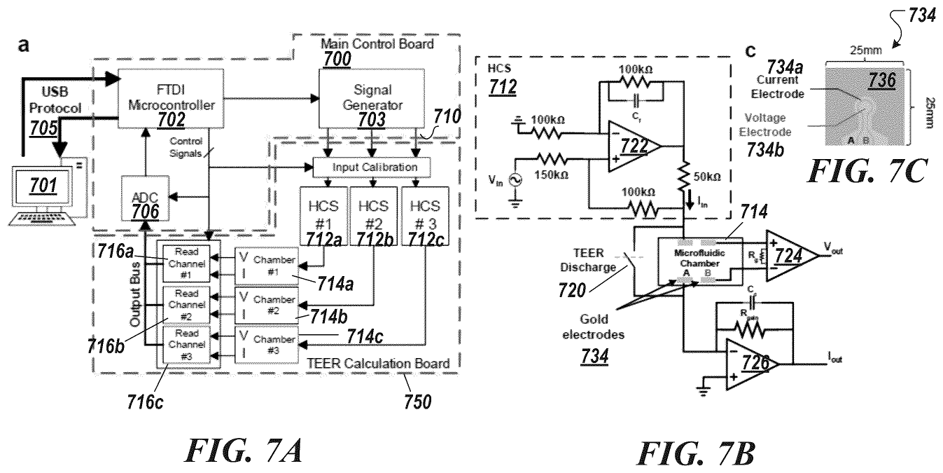

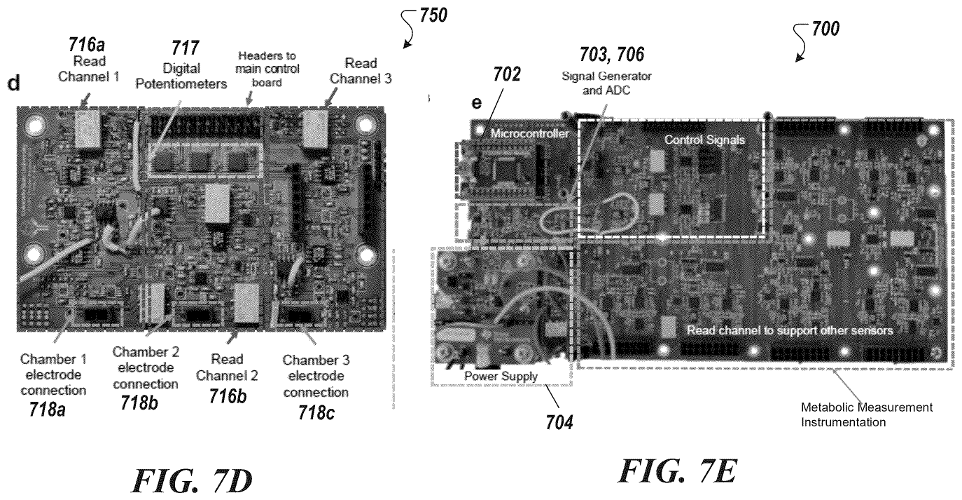

FIG. 8A-8E are graphs demonstrating response signal conditioning and the effect of stimulus signal on TEER calculation.

FIG. 8A shows a raw response signal 802 with unwanted artifacts. The raw data is noisy and also drifts over time because of the offset DC from the HCS. The curve-fitted signal (e.g., 806 in FIG. 8C) removes the unwanted artifacts from the acquired response signal 802.

FIG. 8B is a graph illustrating the drift correction performed by the fitting algorithm. The 1st-degree polynomial 812 (dotted red) of the input data is subtracted point by point from the input raw data 802 (solid red), effectively flattening it out. Once the input data is flattened out, it can be fit to a sinusoidal curve 804 (black).

FIG. 8C is a graph showing the curve fitting algorithm applied to flattened raw data 804 (red) to produce noise-free fitted signal 806 (black).

FIG. 8D is a graph showing a comparison of TEER measured with square wave and 5 kHz sinusoidal stimulus signals. The square wave stimulus is consistently lower than the sinusoidal value.

FIG. 8E is a graph showing the average difference of TEER between sinusoidal and square waveforms for different frequencies of the sinusoidal waveforms, n=10, **: P<0.005.

The sinusoidal curve fitting is necessary to further reduce noise and unwanted artifacts in the acquired TEER signal, as illustrated in FIGS. 8A-8C. The smoothed signal can provide more accurate magnitude and phase values for the subsequent TEER calculation (FIG. 8C). The curve fitting algorithm also provides drifting correction to the acquired voltage response signal. Drifting of the response voltage signal is caused by offset DC current from the Howland circuit. This offset DC current builds up charge on the serial capacitance associated with the electrode's double-layer capacitor, resulting in a constant rate increasing (or decreasing) of the DC voltage at the voltage electrodes from the chamber. This effect can be seen in FIG. 8A. The time-dependent DC shift of the sinusoidal signal in FIG. 8A needs to be leveled before the sinusoidal curve fitting algorithm can be applied to obtain its magnitude and phase. This is done by subtracting a 1st-order polynomial function from the acquired (drifted) voltage signal, as illustrated in FIG. 8B. Accordingly, in some implementations, the proposed system (e.g., measurement circuitry) is configured to perform a data shift operation in order to remove noise from obtained signals (e.g., shift an incoming signal upwards or downwards, apply a curve fitting function, drift correction operation, and/or the like). In so doing, embodiments of the present disclosure are capable of obtaining more accurate measurements from live tissue samples.

The TEER value of an epithelial barrier is the resistance of the transcellular and paracellular pathways combined. However, the TEER values obtained from Eqs. (1) and (2) include additional impedance, such as the electrode double-layer capacitance and the media bulk resistance [3], [9], [30]. In order to obtain the actual TEER values associated with the epithelial barrier, baseline TEER measurements were performed for each experiment to capture the medial bulk resistance. The final TEER value of interest was obtained by subtracting the baseline TEER values from the acquired TEER values. It should be noted that the magnitude |Z| used for TEER measurements should be at an appropriate frequency, not too low where the impedance of the electrode double-layer capacitance dominates, and also not too high where the epithelial layer is shorted by its parallel capacitance, this can be deduced from the equivalent circuit of the epithelial barrier [9]. From the impedance spectrum of the tissue measured with this device, it was found that this value is close to 5 kHz. Similar measurement adjustments may be made for trans-barrier cell electrical resistance measurement.

The TEER value can also be calculated by finding the DC response from a square wave. Since the microfluidic chamber system is also capable of producing a square wave stimulus signal, the TEER using the square waveform stimulus was also calculated. This value shows the pure resistance of the tissue barrier. FIGS. 8D-8E show a set of TEER values obtained using a 5 kHz sinusoidal stimulus vs. a square wave stimulus. It was found that the TEER values obtained using the square waveform are lower (7.2% on average, n=10) than those obtained using the 5 kHz sinusoidal waveform by a constant margin. This is due to the fact that the relatively fast transitions in the square waveform stimulus were able to significantly reduce the effect of the double-layer capacitance associated with the electrodes on TEER magnitudes compared to that from the 5 kHz sinusoidal stimulus. If the sinusoidal stimulus frequency is decreased, then the effect of the double-layer capacitance is more pronounced, making the TEER value increase as the input frequency decreases. FIG. 8E confirms this by showing the percent difference between the sinusoidal and square wave increases as the frequency of the sinusoidal stimulus decreases. When the stimulus frequency reaches 3 kHz and above, the difference between sinusoidal and square wave data flattens out, indicating that the stimulus frequency is now high enough to bypass the double-layer capacitance.

Experiment and Measurement Procedure: After all tissue explants were cut and prepared according to the protocol in Section Animals, Tissue Collection, and Media Preparation, the explants were loaded into the microfluidic chamber, one by one. First, the explants were placed on the bottom half chamber and then gently flattened out using forceps, careful not to touch the luminal side and damage the mucosa. After the tissue was flattened and centered over the holding cavity (FIG. 1B) on the bottom half chamber, the top chamber was slid down the metal screw guides to secure the tissue in place and create a tight seal. The chamber was then tightened using wing nuts and inserted into the card edge connector on the metal enclosure of the system (FIG. 1B). Next, the inlet and outlet tubing were connected. The media outlet tubes were fed into empty glass bottles as a way to determine whether even media outlets from each side of the tissue were achieved during experiments. To remove any air bubbles in the chamber, the media was purged into the chamber at an increased rate (25,000 μL hr⁻¹) for 45 seconds at the start of each experiment. After the initial purge, the media flow rate was reduced to 250 μL hr⁻¹ throughout the experiment. The chambers, media, and the system enclosure are all kept in an incubator set to 37° C.

Live tissue experiments ranged from 24 h-72 h, and a TEER measurement was performed every 2 hours. This created a timeline of the tissues' TEER values to examine the TEER changes as a function of time. For each TEER measurement, the input AC current magnitude was set at 85 μA, and the frequency was swept from 12 Hz to 5 kHz at 20 different frequency points. At each measurement point, the TEER was measured using the sinusoidal waveform stimulus as well as the square waveform stimulus. After the experiment was completed, the chambers were disconnected from all tubing and disconnected from card edge connector. The chamber was then opened to expose the tissue sample and the tissue was preserved following the steps outlined in Section Tissue Sectioning and Histochemistry above.

### Results and Discussion

FIG. 8F-8K are graphs demonstrating the example system's electrical performance. The frequency response for each component in the signal path (a-d) was used to find the system's bandwidth. FIG. 8F shows the input level shifter frequency response. FIG. 8G shows the HCS frequency response. FIG. 8H shows the INA frequency response. FIG. 8I shows the TIA frequency response. In FIG. 8I, the limiting component is the TIA, which sets the system's bandwidth at 47.5 kHz. FIG. 8J shows the read channel noise power spectral density (PSD), which was found to classify the noise specification of the system. FIG. 8K shows the step response of the voltage and current stages in the read channel. The lack of ringing in both step responses confirms the stability of the read channel.

Results of System Electrical and Noise Performance: The frequency response of each circuit component along the signal path is shown in FIGS. 8F-8K. The component with the lowest bandwidth of 47.5 kHz is the transimpedance amplifier TIA (FIG. 8I). The bandwidth of the TIA was set by a compensation capacitor to be roughly ten times higher than the highest frequency of input sinusoidal stimulus (5 kHz). This is to not attenuate any important read channel signals while also filtering out as much high-frequency noise as possible. It should be noted that the TIA tends to have high input inferred noise due to the high thermal noise of its gain-setting resistors. It also sits relatively late in the analog signal chain and its low bandwidth can filter out the output noise from other components (input level shifter and HCS) before it in the signal chain. This sets the full systems bandwidth at 47.5 kHz as intended.

The stability of each read channel is examined by its step response to obtain sufficiently damped responses (FIG. 8K). The noise power spectral density (PSD) of the read channel was measured and shown in FIG. 8J. The results show the total noise power to be 0.126 μV2, well below the minimum output signal power of 82.1 μV2 of the system, resulting in a signal-to-noise ratio (SNR) of 28.14 dB. Other system performance parameters, such as power consumption, TEER measurement error, and the acceptable TEER range with an error less than 5%, were also measured and calculated. Table 1 below summarizes the system-level electrical performance of the microphysiological system.

TABLE 1
System-Level Electrical Performance
of the Microphysiological System

| | | |
|---|---|---|
| Specification | Value | Unit |

Impedance Calculation

| | | |
|---|---|---|
| Frequency Range | 10-5k | Hz |
| Impedance Range (Error <5%) | 150-6.5k | Ω |

Sampling

| | | |
|---|---|---|
| ADC Sampling Rate | 806.4k | samples/s |
| Resolution | 12 | Bits |

Power Consumption

| | | |
|---|---|---|
| V_{dd} | ±5 | V |
| Full system | 4.203 | W |
| TEER Circuit Add-on | 1.17 | W |

Signal Processing

| | | |
|---|---|---|
| Bandwidth | 47.5 | kHz |
| Howland Offset Current (DC) | 1.87 | μA |
| Voltage Gain | 9.8214 | gain |
| Current Gain | 27,000 | gain |

Noise

| | | |
|---|---|---|
| Signal-to-noise ratio (SNR) | 28.14 | dB |
| Total Noise Power | 0.1261 | μV² |
| Average Spectral Density | 623.824 | nV/\sqrt{Hz} |
| Spot Noise at 100 Hz | 1685.79 | nV/\sqrt{Hz} |
| Spot Noise at 1 kHz | 630.688 | nV/\sqrt{Hz} |
| Spot Noise at 10 kHz | 762.691 | nV/\sqrt{Hz} |

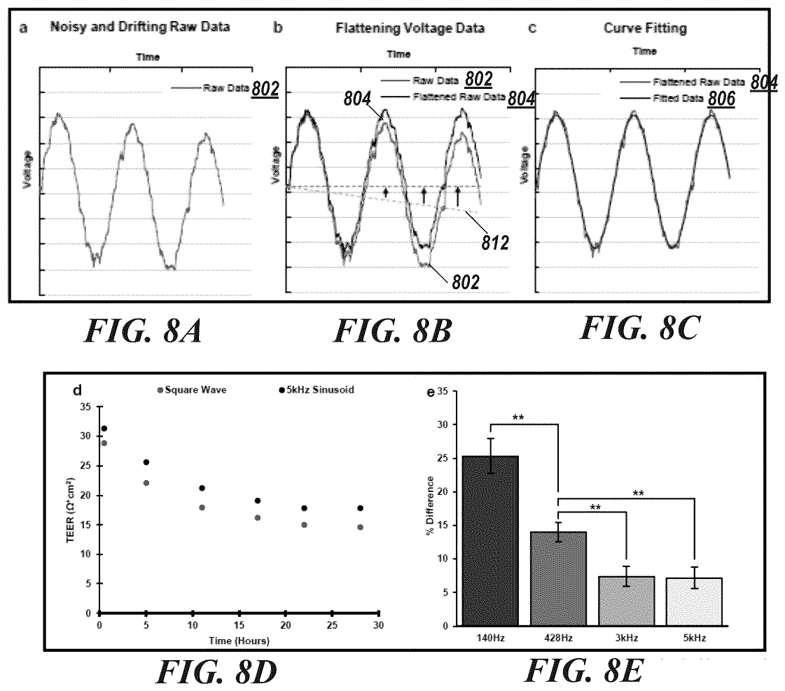

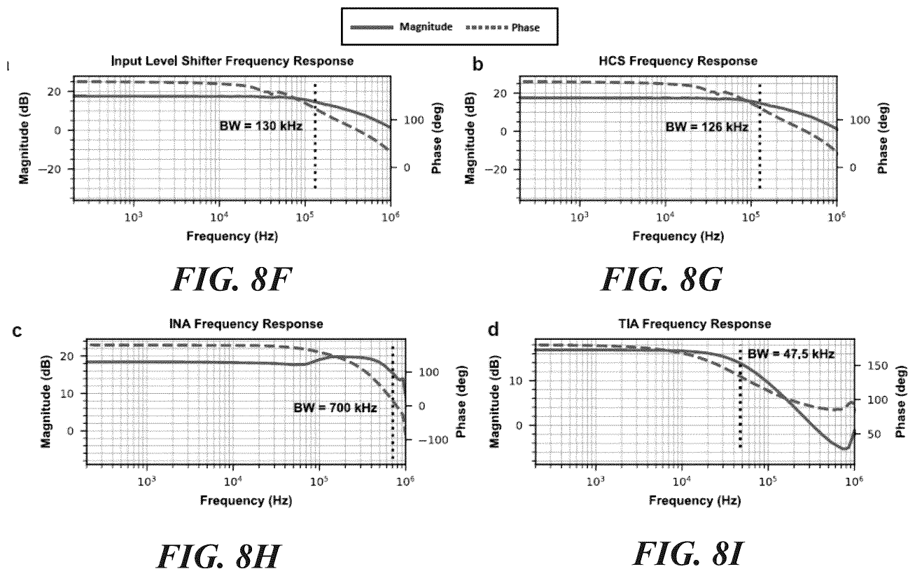

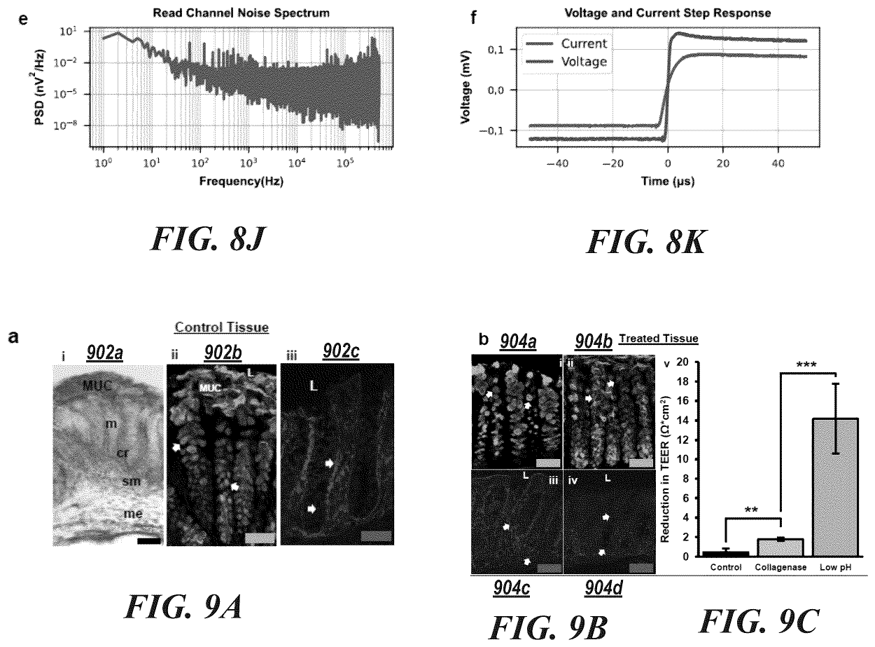

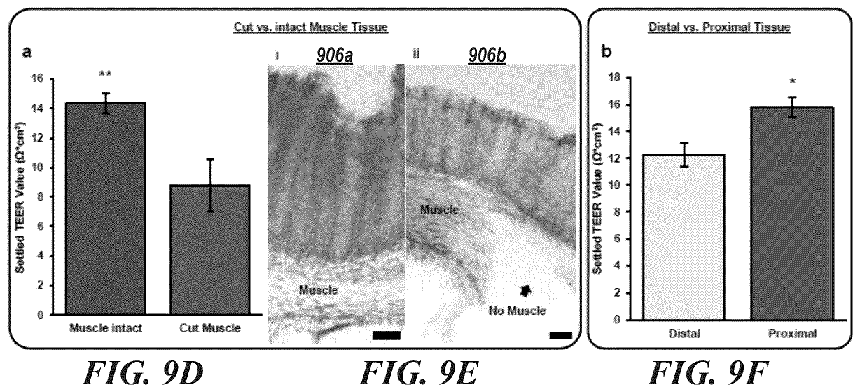

FIG. 9A-9C illustrate results demonstrating that tissue health was maintained for over 72 h in the device and monitored after media treatment. FIG. 9A shows results for a control tissue after the 72 h experiment was conducted. A first image 902a shows Tol blue staining showing maintenance of colon morphology. A second image 902b shows UEA-1+ material confirming the maintenance of epithelial cells and mucus layer. A third image 902c shows Claudin-1 immunoreactivity shows the maintenance of tight junctions between epithelial cells and crypts indicated by claudin-1 immunoreactivity. MUC=mucus layer, m=mucosa, cr=crypt, sm=submucosa, me=muscularis externa, scale bars are 50 μm for B and C. FIG. 9B shows Collagenase treated and acidic luminal media resulted in alterations in goblet cell morphology and tight junction expression indicative of increased barrier permeability. In FIG. 9B, a first image 904a shows Goblet cells labeled with UEA-1 become circular after collagenase treatment. A second image 904b shows acidic media resulting in loss of goblet cell shape and sloughing off of cells near the lumen. A third image 904c shows alterations in tight junction protein expression (claudin-1) following collagenase treatment. A fourth image 904d shows Claudin-1 expression decreased considerably with exposure to acidic media, indicative of substantial barrier disruption.

FIG. 9C is a bar graph showing a distinct reduction in TEER after exposure to different media composition. The difference in TEER was measured from 24 to 48-hour mark after the tissue was enclosed in the device, with the media change occurring at 24 hours. The three media compositions consist of a control media, collagenase-treated media, and low pH media (more details about media composition in Animals, Tissue Collection, and Media Preparation section). L=lumen, scale bars=50 μm, TEER values are normalized to the membrane surface area of the chamber, 0.0314 cm2. Control: n=4, Collagenase: n=10, Low pH: n=3, **: p<0.005; ***: p<0.0001.

Tissue Viability: Tissue health was maintained in the device with barrier integrity over 72 h. Colon explants maintained proper arrangement of mucosal, submucosal, muscular layers, and patterned crypts (902a in FIG. 9A). To protect the body from potential pathogens, healthy intestinal tissue may maintain sophisticated epithelial and mucosal barriers. Specialized epithelial cells, known as goblet cells, are crucial to barrier maintenance as they are responsible for producing and secreting mucin. Goblet cells were characterized due to their essential roles in the maintenance of the barrier. Goblet cell mucopolysaccharides were identified by binding Ulex europaeus agglutinin I (UEA-1) conjugated to rhodamine. After 72 h in the microphysiological system, goblet cells retained their distinct shape (902b in FIG. 9A, arrows), and the inner mucus layer remained intact, confirming maintenance of the mucosal barrier (902b in FIG. 9A). To further verify barrier integrity, tight junctions were examined. Tight junctions adhere epithelial cells together forming a physical barrier between cells to prevent unwanted passage of ions and molecules between epithelial cells. Claudins are a specific type of tight junction protein that helps form the backbone of tight junctions. Claudin-1 is widely expressed in the intestinal epithelium and has essential roles in tight junction integrity. After 72 h in the device, clear claudin-1 immunoreactivity remained around epithelial cells (902c in FIG. 9A), further indicating maintenance of tissue health and barrier integrity.

Using TEER to measure changes in barrier permeability: Changes in TEER were correlated with physiological signs of barrier impairment, such as alterations to epithelial cells, the mucus layer, and tight junction proteins. To induce a disruption to barrier permeability, the luminal side of colon tissue was treated with collagenase or acidic media. Bacterial collagenases are enzymes secreted by endogenous bacteria in the intestines that degrade collagen. Increased collagenase can break down tight junctions between epithelial cells, as well as break down the extracellular matrix of epithelial cells [32]. This leads to increased intestinal permeability, and provides a model for the development of leaky gut syndrome [33]. It has been previously shown that bacterial collagenase in luminal media can be used as a model to create leaky gut by disrupting epithelial cell (goblet cell) morphology and decreasing tight junction (claudin-1) expression [11]. Increased barrier permeability was shown by an increased reduction in TEER with collagenase treatment over time (FIG. 9C). To confirm that reductions in TEER correlated with physiological characteristics of increased intestinal permeability, goblet cells and claudin-1 were examined. Following collagenase treatment, goblet cells became more circular in shape (904a in FIG. 9B), and claudin-1 immunoreactivity was moderately decreased (904c in FIG. 9B), indicating barrier impairment.

To test whether changes in TEER matched changes in physiological changes, acidic media (pH 2) was added to the luminal side of the tissue to induce significant damage to the intestinal barrier. Cells need to maintain a pH of 7.4 to function properly. Lowering the pH to 2.0 leads to significant epithelial cell death and alterations in cellular processes, creating drastic increases in permeability. This was confirmed with goblet cells losing distinct shape and sloughing off near the lumen (904b in FIG. 9B). Claudin-1 immunoreactivity dramatically decreased, indicating substantial loss of tight junctions (904d in FIG. 9B). These dramatic changes in epithelial cell and claudin-1 morphology correlate with the significant reduction of TEER following acidic pH treatment (FIG. 9C).

Differences in tissue explant detected by TEER. FIG. 9D-9F are results demonstrating physical differences in tissue explant. FIG. 9D is a bar chart showing the difference in settled TEER value of tissue explants with the muscle intact vs. with the muscle removed. The settled TEER value is taken 24 hours after the tissue is enclosed in the chamber. A first image 906a in FIG. 9E shows Tol blue staining of tissue with intact muscle. A second image 906b in FIG. 9E shows Tol blue staining tissue with muscle removed. FIG. 9F is a graph showing settled TEER values for different regions of mouse colon tissue. Proximal tissue was defined as the three pieces of tissue closest to the cecum, and distal tissue was defined as the two pieces farthest from the cecum and closest to the rectum. Each tissue piece was approximately 5 mm in length. The settled TEER value was taken approximately 24 hours after the tissue had been enclosed in the device. Muscle intact: n=15, Cut muscle: n=7, Distal: n=6, Proximal: n=9, **: P<0.005, *: P<0.01.

Cut muscle vs. muscle intact muscle: To determine whether distinct tissue components contributed differentially to TEER, the muscle layer was dissected away. Thereby removing the muscularis externa, a major subepithelial structure of the colon. TEER was measured after 24 h inside the chamber, allowing sufficient time for the tissue to equilibrate to its new environment. Removal of the muscle layer decreased TEER by about 39% (FIG. 9D). This result is consistent with previous reports that have performed experiments to study the contribution of sub-epithelial resistance. The values reported have ranged from 15% to 80% of the total epithelial resistance concentrated in the sub-epithelium, depending on the location in the intestine as well as the animal [8, 30, 34-36]. Research using rat jejunum has shown a much larger contribution to total resistance done by the sub-epithelium (78-80%) [8, 34]. Whereas measurements on the ileum, colon, and rectum in both rats and mice have shown much lower contributions (15-45%) [30,35,36]. This is consistent with the results found here using a mouse colon. Confirmation of total muscle dissection was done by Toluidine blue staining, as seen in image 906b in FIG. 9E when compared to tissue with the muscle intact, shown in image 906a in FIG. 9E. Demonstrating the TEER calculated with the muscle dissected is an accurate representation of the epithelial resistance alone and has little to no contribution from subepithelial resistances. This demonstration is lacking in all previous research studying the contribution of subepithelial resistance by subepithelial stripping or dissection [34-36]. These images, alongside the TEER measurements, provide new evidence for the contribution of subepithelial resistance to total epithelial resistance.

Proximal vs. Distal Colon: Since the distal colon is generally thicker than the proximal colon, the study investigated if these differences in tissue thickness correlated with changes in TEER. Based on the results that an intact muscle layer increased TEER (FIG. 9D), the study expected TEER to be higher in the distal colon. However, the study observed about a 19% decrease in TEER in the distal colon compared to the proximal colon (FIG. 9F). Interestingly, this is consistent with previous reports showing higher TEER values in mouse proximal colon compared to mid or distal colon [6]. Based on the observed effect of muscle removal, the study found it likely that other factors contribute to differences in TEER between proximal and distal colon. One possible factor is luminal pH. The pH of the proximal colon is typically around 5.8-6.5, while the distal colon is typically around 7-7.637. Previous studies have attributed increased TEER to decreased pH in the culture media [38]. This is consistent with our observations that an empty chamber filled with acidic media (pH 2) had higher TEER than control media (pH 7.4).

Higher TEER in the proximal colon may be a function of the thickness of the mucus layer. The colon houses the majority of the intestinal microbiome and, therefore, has a thick mucus layer that physically separates bacteria from underlying epithelial cells. The proximal colon has been reported to have a thicker mucus layer compared to the distal colon, with an increased number and size of goblet cells, as well as increased expression of mucin-239. In vivo measurements of the mouse colon have estimated the colon mucus layer to be ˜190 μm40. This is significant as the total tissue thickness of the mouse colon is estimated to be around 140-300 μm41. The mucus layer may have a profound effect on TEER; however, studies investigating the contribution of the mucus layer on TEER are lacking. The mucus layer can be easily washed off in tissue dissection and preparation.

Example System Performance. Besides the electrical performance metrics and the experimental results shown above, some unique capabilities of the microphysiological system are compared with the existing systems/devices reported in the literature as illustrated in Table 2 below. Compared to the existing systems/devices, this system is able to maintain longer tissue viability of intestine tissue with integrated electrodes to provide real-time TEER measurements. The custom electronics and system design also provide experiment configuration and improved throughput.

TABLE 2
Comparison of Epithelial Barrier Investigation Devices

| | Biological Sample | | | | | | |
|---|---|---|---|---|---|---|---|
| | | Demonstrated | | Electrical Permeability | | | |
| | Sample | Tissue | | TEER | Electrode | Stimulus | Measurement |
| | type | Viability | | capable? | Type | Signal | Electronics |
| Transwell | Cell | — | | Yes | Ag/AgCL | DC | Commercial |
| [2], [42] | monolayer | | | | “stick” | | Benchtop |
| | | | | | electrodes | | |
| Liang et al., | Cell | — | | Yes | Integrated glass | Up to | Commercial |
| 2023 [12] | monolayer | | | | chip | 10 MHz | Benchtop |
| | (canine | | | | | | |
| | kidney) | | | | | | |
| Helm et al., | Cell | — | | Yes | Polycarbonate | Up to | Commercial |
| 2019 [19] | monolayer | | | | substrate | 100 kHz | Benchtop |
| | (Caco-2) | | | | electrode chips | | |
| Fernandes | Cell | — | | Yes | Integrated glass | Up to | Custom-built |
| et al., 2022 | monolayer | | | | chip | 100 kHz | |
| [20] | (GI tract | | | | | | |
| | and airway) | | | | | | |
| Navicyte | Mouse and | <3 | h | Yes | Ag/AgCL | DC | Commercial |
| [6] | human | | | | “stick” | | Benchtop |
| | intestinal | | | | electrodes | | |
| | tissue | | | | | | |
| Clarke et | Mouse | 3 | h | Yes | Ag/AgCL | DC | Commercial |
| al., 2009 | colon | | | | electrodes | | Benchtop |
| [5] | tissue | | | | connected by | | |
| | | | | | salt bridge | | |
| Calvo et al., | Frog | — | | Yes | Integrated | Up to | Custom-built |
| 2020 [23] | epithelial | | | | “stick” | 100 kHz | |
| | tissue | | | | electrodes | | |
| Dawson et | Human | 72 | h | No | — | — | — |
| al., 2016 | intestinal | | | | | | |
| [43] | tissue | | | | | | |
| Poenar et | Porcine | 48 | h | Yes | Integrated | DC | Commercial |
| al., 2020 | esophageal | | | | “stick” | | Benchtop |
| [7] | tissue | | | | electrodes | | |
| Cherwin et | Mouse | 72 | h | No | — | — | — |
| al., 2023 | colon | | | | | | |
| [11] and | tissue | | | | | | |
| Richardson | | | | | | | |
| et al., 2020 | | | | | | | |
| [14] | | | | | | | |
| Amiraabadi | Porcine | 24 | h | No | Optical Fiber | — | — |
| et al., 2022 | and human | | | | Sensor | | |
| [44] | colon tissue | | | | | | |
| This study | Mouse | 72 | h | Yes | Integrated glass | Up to | Custom-built |
| | colon | | | | chip | 5 kHz | |
| | tissue | | | | | | |

| | Biological Sample | | | | |
|---|---|---|---|---|---|
| | | Demonstrated | | Chamber/System Design | |
| | Sample | Tissue | | Microfluidic | |
| | type | Viability | | Support | Throughput |
| Transwell | Cell | — | | No | 96 |
| [2], [42] | monolayer | | | | |
| Liang et al., | Cell | — | | Yes | 1 |
| 2023 [12] | monolayer | | | | |
| Helm et al., | (canine | | | | |
| 2019 [19] | kidney) | | | | |
| Fernandes | Cell | — | | Yes | 1 |
| et al., 2022 | monolayer | | | | |
| [20] | (Caco-2) | | | | |
| | Cell | — | | One side | 8 |
| | monolayer | | | only | |
| | (GI tract | | | | |
| | and | | | | |
| | airway) | | | | |
| Navicyte [6] | Mouse and | <3 | h | No | 6 |
| | human | | | | |
| | intestinal | | | | |
| | tissue | | | | |
| Clarke et | Mouse | 3 | h | No | 1 |
| al., 2009 [5] | colon | | | | |
| | tissue | | | | |
| Calvo et al., | Frog | — | | No | 1 |
| 2020 [23] | epithelial | | | | |
| | tissue | | | | |
| Dawson et | Human | 72 | h | Yes | 1 |
| al., 2016 | intestinal | | | | |
| [43] | tissue | | | | |
| Poenar et | Porcine | 48 | h | Yes | 1 |
| al., 2020 [7] | esophageal | | | | |
| | tissue | | | | |
| Cherwin et | Mouse | 72 | h | Yes | 1 |
| al., 2023 | colon | | | | |
| [11] and | tissue | | | | |
| Richardson | Porcine | 24 | h | Yes | 1 |
| et al., 2020 | and human | | | | |
| [14] | colon | | | | |
| Amiraabadi | tissue | | | | |
| et al., 2022 | | | | | |
| [44] | | | | | |
| This study | Mouse | 72 | h | Yes | 3 |
| | colon | | | | |
| | tissue | | | | |

### Discussion

Embodiments of the present disclosure present a highly integrated microphysiological system for studying the live tissue barrier permeability of the mouse colon. The unique design of the microfluidic chamber is capable of securing an explant of mouse colon tissue between two independent media pathways creating a micro-physiological environment inside the chamber comparable to the environment in vivo. The use of proper media provides nutrients, supports the gut microbiome, and creates important oxygen gradients across the tissue to keep tissue viability for an extended period of time. After 72 hours in the chamber, the tissue explants displayed an inner mucus layer, robust goblet cells, and evident tight junction function along the length of the epithelial layer. These characteristics all serve as strong indicators of sustained barrier integrity. This preservation of tissue viability addresses a significant drawback in existing live tissue barrier permeability devices.

Integrated electrode chips allow the microfluidic chamber to successfully characterize barrier permeability using TEER measurements in real-time. The plug-and-play nature of the system design simplifies the experiment setup and allows for all chambers to be reusable and universal. Unlike most existing systems where bulky and expensive benchtop equipment is needed to perform experiments, the integrated support electronics made the overall system small enough to fit into an incubator. Furthermore, architectural scalability allows multiple chambers to be connected to the system, enabling higher throughput of controlled experiments using samples from the same donor. The use of the system is further enhanced by a custom-built GUI, which was developed to allow each experiment to be customizable and run from any host computer. Indeed, the exemplary microphysiological system can be employed in the investigation of barrier health of live tissues. Real-time barrier health measurements are crucial to developing more accurate ex vivo tissue models for studying the health and chemical response of epithelial cells.

### Configuration of Certain Implementations

The construction and arrangement of the systems and methods as shown in the various implementations are illustrative only. Although only a few implementations have been described in detail in this disclosure, many modifications are possible (e.g., variations in sizes, dimensions, structures, shapes, and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the implementations without departing from the scope of the present disclosure.

The present disclosure contemplates methods, systems, and program products on any machine-readable media for accomplishing various operations. The implementations of the present disclosure may be implemented using existing computer processors, or by a special purpose computer processor for an appropriate system, incorporated for this or another purpose, or by a hardwired system. Implementations within the scope of the present disclosure include program products, including machine-readable media for carrying or having machine-executable instructions or data structures stored thereon. Such machine-readable media can be any available media that can be accessed by a computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures, and which can be accessed by a general purpose or special purpose computer or other machine with a processor.

When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general-purpose computer, special purpose computer, or special purpose processing machines to perform a certain function or group of functions.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not. Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

The following patents, applications, and publications, as listed below and throughout this document, describe various applications and systems that could be used in combination the exemplary system and are hereby incorporated by reference in their entirety herein.
[1] Moens, E. & Veldhoen, M. Epithelial barrier biology: good fences make good neighbours. Immunology 135, 1-8 (2012).
[2] Walter, F. R. et al. A versatile lab-on-a-chip tool for modeling biological barriers. Sensors and Actuators B: Chemical 222, 1209-1219 (2016).
[3] Srinivasan, B. et al. TEER Measurement Techniques for In Vitro Barrier Model Systems. SLAS Technology 20, 107-126 (2015).
[4] Ussing, H. H. & Zerahn, K. Active transport of sodium as the source of electric current in the short-circuited isolated frog skin. Acta Physiol Scand 23, 110-127 (1951).
[5] Clarke, L. L. A guide to Ussing chamber studies of mouse intestine. American Journal of Physiology-Gastrointestinal and Liver Physiology 296, G1151-G1166 (2009).
[6] Thomson, A. et al. The Ussing chamber system for measuring intestinal permeability in health and disease. BMC Gastroenterol 19, 98 (2019).
[7] Poenar, D. P., Yang, G., Wan, W. K. & Feng, S. Low-Cost Method and Biochip for Measuring the Trans-Epithelial Electrical Resistance (TEER) of Esophageal Epithelium. Materials (Basel) 13, 2354 (2020).
[8] Fromm, M., Schulzke, J. D. & Hegel, U. Epithelial and subepithelial contributions to transmural electrical resistance of intact rat jejunum, in vitro. Pflugers Arch 405, 400-402 (1985).
[9] Benson, K., Cramer, S. & Galla, H.-J. Impedance-based cell monitoring: barrier properties and beyond. Fluids and Barriers of the CNS 10, 5 (2013).
[10] Leung, C. M. et al. A guide to the organ-on-a-chip. Nat Rev Methods Primers 2, 1-29 (2022).
[11] Cherwin, A. E. et al. Microfluidic organotypic device to test intestinal mucosal barrier permeability ex vivo. Lab Chip 10.1039.D3LC00615H (2023) doi: 10.1039/D3LC00615H.
[12] Liang, F. et al. A microfluidic tool for real-time impedance monitoring of in vitro renal tubular epithelial cell barrier. Sensors and Actuators B: Chemical 392, 134077 (2023).
[13] McLean, I. C., Schwerdtfeger, L. A., Tobet, S. A. & Henry, C. S. Powering ex vivo tissue models in microfluidic systems. Lab Chip 18, 1399-1410 (2018).
[14] Richardson, A. et al. A microfluidic organotypic device for culture of mammalian intestines ex vivo. Anal. Methods 12, 297-303 (2020).
[15] van der Helm, M. W. et al. Direct quantification of transendothelial electrical resistance in organs-on-chips. Biosensors and Bioelectronics 85, 924-929 (2016).
[16] Odijk, M. et al. Measuring direct current trans-epithelial electrical resistance in organ-on-a-chip microsystems. Lab Chip 15, 745-752 (2015).
[17] Yeste, J., Illa, X., Alvarez, M. & Villa, R. Engineering and monitoring cellular barrier models. J Biol Eng 12, 18 (2018).
[18] Thuenauer, R., Rodriguez-Boulan, E. & Römer, W. Microfluidic approaches for epithelial cell layer culture and characterisation. Analyst 139, 3206-3218 (2014).
[19] Helm, M. W. van der et al. Non-invasive sensing of transepithelial barrier function and tissue differentiation in organs-on-chips using impedance spectroscopy. Lab Chip 19, 452-463 (2019).
[20] Fernandes, J. et al. Real-time monitoring of epithelial barrier function by impedance spectroscopy in a microfluidic platform. Lab Chip 22, 2041-2054 (2022).
[21] Danku, A. E., Dulf, E.-H., Braicu, C., Jurj, A. & Berindan-Neagoe, I. Organ-On-A-Chip: A Survey of Technical Results and Problems. Front Bioeng Biotechnol 10, 840674 (2022).
[22] Ghiselli, F., Rossi, B., Piva, A. & Grilli, E. Assessing Intestinal Health. In Vitro and Ex vivo Gut Barrier Models of Farm Animals: Benefits and Limitations. Front Vet Sci 8, 723387 (2021).
[23] Calvo, P. C., Campo, O., Guerra, C., Castaño, S. & Fonthal, F. Design of using chamber system based on electrical impedance spectroscopy (EIS) to measure epithelial tissue. Sensing and Bio-Sensing Research 29, 100357 (2020).
[24] Liu, J. et al. The differential Howland current source with high signal to noise ratio for bioimpedance measurement system. Rev Sci Instrum 85, 055111 (2014).
[25] Obeidat, Y. M. et al. Design of a multi-sensor platform for integrating extracellular acidification rate with multi-metabolite flux measurement for small biological samples. Biosensors and Bioelectronics 133, 39-47 (2019).
[26] Wegener, J., Hakvoort, A. & Galla, H.-J. Barrier function of porcine choroid plexus epithelial cells is modulated by cAMP-dependent pathways in vitro. Brain Research 853, 115-124 (2000).
[27] Wegener, J., Abrams, D., Willenbrink, W., Galla, H.-J. & Janshoff, A. Automated multi-well device to measure transepithelial electrical resistances under physiological conditions. Biotechniques 37, 590, 592-594, 596-597 (2004).
[28] Gijzen, L. et al. An Intestine-on-a-Chip Model of Plug-and-Play Modularity to Study Inflammatory Processes. SLAS Technol 25, 585-597 (2020).
[29] Douville, N. J. et al. Fabrication of two-layered channel system with embedded electrodes to measure resistance across epithelial and endothelial barriers. Anal Chem 82, 2505-2511 (2010).
[30] Gitter, A. H., Fromm, M. & Schulzke, J.-D. Impedance analysis for the determination of epithelial and subepithelial resistance in intestinal tissues. Journal of Biochemical and Biophysical Methods 37, 35-46 (1998).
[31] Gitter, A. H., Schulzke, J.-D., Sorgenfrei, D. & Fromm, M. Ussing chamber for high-frequency transmural impedance analysis of epithelial tissues. Journal of Biochemical and Biophysical Methods 35, 81-88 (1997).
[32] Duarte, A. S., Correia, A. & Esteves, A. C. Bacterial collagenases—A review. Crit Rev Microbiol 42, 106-126 (2016).
[33] Aleman, R. S., Moncada, M. & Aryana, K. J. Leaky Gut and the Ingredients That Help Treat It: A Review. Molecules 28, 619 (2023).
[34] Hemlin, M., Jodal, M., Lundgren, O., Sjövall, H. & Stage, L. The importance of the subepithelial resistance for the electrical properties of the rat jejunum in vitro. Acta Physiologica Scandinavica 134, 79-88 (1988).
[35] Tai, Y.-H. & Tai, C.-Y. The conventional short-circuiting technique under-short-circuits most epithelia. J. Membrain Biol. 59, 173-177 (1981).
[36] Gitter, A. H., Bendfeldt, K., Schulzke, J. D. & Fromm, M. Trans/paracellular, surface/crypt, and epithelial/subepithelial resistances of mammalian colonic epithelia. Pflugers Arch-Eur J Physiol 439, 477-482 (2000).
[37] Mikolajczyk, A. E., Watson, S., Surma, B. L. & Rubin, D. T. Assessment of Tandem Measurements of pH and Total Gut Transit Time in Healthy Volunteers. Clinical and Translational Gastroenterology 6, e100 (2015).
[38] Helms, H. C., Waagepetersen, H. S., Nielsen, C. U. & Brodin, B. Paracellular tightness and claudin-5 expression is increased in the BCEC/astrocyte blood-brain barrier model by increasing media buffer capacity during growth. AAPS J 12, 759-770 (2010).
[39] Bergstrom, K. et al. Proximal colon-derived O-glycosylated mucus encapsulates and modulates the microbiota. Science 370, 467-472 (2020).
[40] Hugenholtz, F. & De Vos, W. M. Mouse models for human intestinal microbiota research: a critical evaluation. Cell. Mol. Life Sci. 75, 149-160 (2018).
[41] Abdelrahman, M. A. et al. High-Frequency Ultrasound for In Vivo Measurement of Colon Wall Thickness in Mice. Ultrasound in Medicine & Biology 38, 432-442 (2012).
[42] Leonard, F., Collnot, E.-M. & Lehr, C.-M. A three-dimensional coculture of enterocytes, monocytes and dendritic cells to model inflamed intestinal mucosa in vitro. Mol Pharm 7, 2103-2119 (2010).
[43] Dawson, A. et al. A microfluidic chip based model for the study of full thickness human intestinal tissue using dual flow. Biomicrofluidics 10, 064101 (2016).
[44] Eslami Amirabadi, H. et al. Intestinal explant barrier chip: long-term intestinal absorption screening in a novel microphysiological system using tissue explants. Lab Chip 22, 326-342 (2022).

What is claimed is:

1. A device comprising:
a first chamber body;
a second chamber body having a correspondence to the first chamber body and is configured to fixably couple to the first chamber body to form an assembled chamber,
wherein the first chamber body defines a first elongated-curved internal structure, and wherein the second chamber body defines a second elongated-curved internal structure that, together with the first internal structure, defines an elongated-curved chamber volume to hold a tissue sample and to promote laminar flow when media is flowing therethrough,
wherein the first chamber body and the second chamber body each define (i) a first fluidic channel having smooth flow paths from a first inlet to the elongated-curved chamber volume and from the elongated-curved chamber volume to a first outlet to transport a first media from the first inlet to the first outlet through a first region of the elongated-curved chamber volume and (ii) a second fluidic channel having smooth flow paths from a second inlet to the elongated-curved chamber volume and from the elongated-curved chamber volume to a second outlet to transport a second media from the second inlet to the second outlet through a second region of the elongated-curved chamber volume,
wherein the first internal structure comprises a plurality of fixation elements extending therefrom, wherein the second internal structure comprises corresponding recesses for the fixation elements, wherein the plurality of fixation elements and corresponding recesses provide for fixation of the tissue in the first internal structure and the second internal structure, to provide separation of the first region and the second region.

2. The device of claim 1 further comprising:
a first set of electrodes embedded within the first chamber body; and
a second set of electrodes embedded within the second chamber body.

3. The device of claim 2 further comprising:
a first electronic circuit board having measurement circuitries to couple to the first set of electrodes; and
a second electronic circuit board having measurement circuitries to couple to the second set of electrodes,
wherein the first electronic circuit board is fixably coupled to the first chamber body, and wherein the second electronic circuit board is fixably coupled to the second chamber body.

4. The device of claim 3, further comprising:
a third electronic circuit board having a first interface to the first electronic circuit board, wherein the third electronic circuit board includes a controller to execute a measurement program.

5. The device of claim 3, wherein the first set of electrodes is embedded in a first electrode chip that is operatively coupled to the measurement circuitries of the first electronic circuit, wherein the first electrode chip is positioned within the first region to obtain a first plurality of measurements from a first side of the tissue when inside the device; and
wherein the second set of electrodes is embedded in a second electrode chip that is operatively coupled to the measurement circuitries of the second electronic circuit, wherein the second electrode chip is positioned within the second region to obtain a second plurality of measurements from a second side of the tissue when inside the device.

6. The device of claim 5, wherein the first electrode chip and the second electrode chip each comprise at least one voltage electrode and at least one current electrode for obtaining the first plurality of measurements and the second plurality of measurements.

7. The device of claim 5, wherein the first electrode chip and the second electrode chip each comprise a Trans-Epithelial Electrical Resistance (TEER) sensor positioned in proximity to a respective side of the tissue sample within the assembled chamber.

8. The device of claim 5, wherein the first electrode chip and the second electrode chip are each configured to measure a Trans-Epithelial Electrical Resistance and at least one of pH, oxygen level, glucose level, and lactose level in relation to a respective side of the tissue sample.

9. The device of claim 8, wherein the device is further configured with pairs of pH, O₂, glucose, and/or lactate sensors at each respective side of the tissue sample, including at least a first side and a second side of the tissue sample, configured to measure metabolites in the media in order to determine a difference in metabolic activity.

10. The device of claim 1, wherein the elongated-curved chamber volume comprises a central portion, a front distal portion, and a rear distal portion, wherein the central portion is wider than the front and rear distal portions.

11. The device of claim 1, wherein the first inlet and first outlet are positioned at a top position of the first chamber body, and wherein the second inlet and second outlet are positioned at a top position of the second chamber body, wherein the first outlet is positioned relatively above the first elongated-curved internal structure of the first chamber body to guide air bubble flow to the first outlet, and wherein the first outlet is positioned relatively above the first elongated-curved internal structure of the first chamber body to guide air bubble flow to the second outlet.

12. The device of claim 1, wherein the plurality of fixation elements are tapered.

13. The device of claim 1, wherein the plurality of fixation elements are arranged in a ring, a set of concentric rings, or arranged in a non-uniform pattern.

14. The device of claim 1, wherein the assembled chamber is configured as an Ussing chamber.

15. The device of claim 4, wherein the controller is configured to determine one or more properties of each tissue sample based, at least in part, on a difference between a first plurality of measurements and a second plurality of measurements.

16. The device of claim 4, wherein the controller is configured to perform a data shift operation and/or drift correction on obtained measurements.

17. The device of claim 4, wherein the controller is operatively coupled to a display or hosting web service for outputting obtained measurements.

18. The device of claim 3, wherein the first electronic circuit board and the second electronic circuit board each comprise front-end mixed signal acquisition circuitries for the respective set of electrodes.

19. The device of claim 1, wherein the first inlet has an axis that is in-line with that of the first outlet along a first plane, wherein the second inlet has an axis that is in-line with that of the second outlet along a second plane, and wherein the first plane and the second plane are parallel to provide a generally in-line configuration of the first inlet, first outlet, second inlet, and second outlet for the device.

20. A test system comprising:
at least one device, the at least one device comprising:
a first chamber body;
a second chamber body having a correspondence to the first chamber body and is configured to fixably couple to the first chamber body to form an assembled chamber,
wherein the first chamber body defines a first elongated-curved internal structure, and wherein the second chamber body defines a second elongated-curved internal structure that, together with the first internal structure, defines an elongated-curved chamber volume to hold a tissue sample and to promote laminar flow when media is flowing therethrough,
wherein the first chamber body and the second chamber body each define (i) a first fluidic channel having smooth flow paths from a first inlet to the elongated-curved chamber volume and from the elongated-curved chamber volume to a first outlet to transport a first media from the first inlet to the first outlet through a first region of the elongated-curved chamber volume and (ii) a second fluidic channel having smooth flow paths from a second inlet to the elongated-curved chamber volume and from the elongated-curved chamber volume to a second outlet to transport a second media from the second inlet to the second outlet through a second region of the elongated-curved chamber volume,
wherein the first internal structure comprises a plurality of fixation elements extending therefrom, wherein the second internal structure comprises corresponding recesses for the fixation elements, wherein the plurality of fixation elements and corresponding recesses provide for fixation of the tissue in the first internal structure and the second internal structure, to provide separation of the first region and the second region.
